(19) United States
(12) Patent Application Publication
(10) Pub. No.: US 2021/0290086 A1
(43) Pub. Date: Sep. 23, 2021

(54) BLOOD PRESSURE MEASUREMENT DEVICE

(71) Applicants: OMRON Corporation, Kyoto (JP); OMRON HEALTHCARE Co., Ltd., Kyoto (JP)

(72) Inventor: Tomoyuki NISHIDA, Kyoto (JP)

(21) Appl. No.: 17/303,813

(22) Filed: Jun. 8, 2021

Related U.S. Application Data
(63) Continuation of application No. PCT/JP2019/047165, filed on Dec. 3, 2019.

(30) Foreign Application Priority Data
Dec. 13, 2018 (JP) 2018-233415

(51) Int. Cl. A61B 5/021; A61B 5/022; A61B 5/00

(52) U.S. Cl. CPC A61B 5/02141; A61B 5/6824; A61B 5/02233

(57) ABSTRACT

A blood pressure measurement device comprising, a case, a cuff structure connected to the case and configured to be inflated with a fluid, a curler curving to follow a circumferential direction of a site of attachment to a living body, the curler being formed with a first end and a second end spaced apart from each other, the cuff structure being provided on the curler, a flow path constituted by at least one of the case or the cuff structure, the flow path being configured to communicate an inside and an outside of the case and allow flow of air, and a water-repellent portion provided on at least one of an inner surface of the flow path or a portion of an outer surface of the at least one of the case or the cuff structure that constitutes the flow path, the portion being continuous with the flow path.

### CROSS-REFERENCE TO RELATED APPLICATIONS

This application is the U.S. national stage application filed pursuant to 35 U.S.C. 365(c) and 120 as a continuation of International Patent Application No. PCT/JP2019/047165, filed Dec. 3, 2019, which application claims priority from Japanese Patent Application No. 2018-233415, filed Dec. 13, 2018, which applications are incorporated herein by reference in their entireties.

### TECHNICAL FIELD

The present invention relates to a blood pressure measurement device for measuring blood pressure.

### BACKGROUND

In recent years, blood pressure measurement devices for measuring blood pressure are being used to monitor health status at home, as well as in medical facilities. A blood pressure measurement device detects vibration of the artery wall to measure blood pressure by, for example, inflating and deflating a cuff wrapped around the upper arm or the wrist of a living body and detecting the pressure of the cuff using a pressure sensor.

As such a blood pressure measurement device, for example, a so-called integral type is known in which a cuff and a device body feeding a fluid to the cuff are integrally configured as disclosed in JP 2018-102743 A (for example, refer to Patent Document 1).

In such a blood pressure measurement device, a pump is accommodated in a case of the device body. Furthermore, when a suction port of the pump is configured to open inside the case, a ventilation hole for introducing air residing outside the case into the case is formed in the case. The pump draws and compresses air residing inside the case and feeds the air to the cuff. When the pump draws the air residing inside the case and the case interior becomes negative in pressure, the air outside the case is fed into the case through the ventilation hole.

### CITATION LIST

### Patent Literature

Patent Document 1: JP 2018-102743 A

### SUMMARY OF INVENTION

### Technical Problem

For the above-described blood pressure measurement device, wearable devices attached to the wrist have recently been considered. With a blood pressure measurement device configured as a wearable device, sweat of a user readily adheres to the blood pressure measurement device. Furthermore, presumably, the user contacts water with the blood pressure measurement device attached in many occasions.

When the sweat of the user or water outside the blood pressure measurement device comes into contact with the blood pressure measurement device, there is a risk that the sweat or the water may enter the case through the ventilation hole. Further, when the case is constituted by a combination of a plurality of materials, there is a risk that the sweat or the water may enter the case due to a capillary phenomenon.

Thus, an object of the present invention is to provide a blood pressure measurement device that can improve waterproof performance.

### Solution

According to one aspect, there is provided a blood pressure measurement device including a case, a cuff structure connected to the case and configured to be inflated with a fluid, a flow path constituted by at least one of the case or the cuff structure, the flow path being configured to communicate an inside and an outside of the case and allow flow of air, and a water-repellent portion provided on at least one of an inner surface of the flow path or a portion of an outer circumferential surface of the at least one of the case or the cuff structure that constitutes the flow path, the portion being continuous with the flow path.

Here, the fluid includes a liquid and air. The cuff refers to a member that is wrapped around the upper arm, the wrist, or the like of a living body when the blood pressure is measured and that is inflated by being fed with the fluid. The cuff includes a bag-like structure such as an air bag.

The water-repellent portion is formed by applying a water-repellent treatment. The term “water-repellent treatment” refers to a treatment that increases a contact angle of water compared to that before the water-repellent treatment is applied. The water-repellent treatment is preferably a treatment that makes the contact angle of water 90 degrees or greater. As an example of the water-repellent treatment, a fluorine treatment is applied. A fluorine treatment is applied by, for example, applying a treatment liquid containing fluororesin using a brush or a cotton swab. The treatment liquid is dried, thereby forming a film containing the fluororesin. This film serves as the water-repellent portion. Alternatively, the water-repellent portion may be configured by forming a member constituting the flow path from a material having a desired water repellency. Alternatively, of the member constituting the flow path, only a portion constituting the flow path may be formed from a material having a desired water repellency to configure the water-repellent portion.

According to this aspect, entry of water from outside the case into the flow path is suppressed by an action of pushing the water that is about to enter the flow path to outside the flow path, which is generated by the water-repellent portion provided on the inner surface of the flow path. Further, the water-repellent portion provided on a portion of the outer surface of a member constituting the flow path, the portion being continuous with the flow path, suppresses adherence of the water to the portion. With the entry of water into the flow path being suppressed due to any of these effects, the entry of water into the case is suppressed. As a result, the water resistance of the blood pressure measurement device is improved.

In the blood pressure measurement device according to the one aspect described above, there is provided a blood pressure measurement device in which the case includes an outer case having a tubular shape, and the flow path is a ventilation hole including a first open end and a second open end, the first open end being disposed on an inner circumferential surface of the outer case, the second open end being disposed on an outer circumference of the outer case, the second open end being disposed on a living body side in an axial direction of the outer case with respect to the first open end.

According to this aspect, the blood pressure measurement device is set into a posture in which the axial direction of the outer case is parallel with a gravitational direction, and thus the second open end is disposed below the first open end in the gravitational direction, thereby the water entering the ventilation hole can move out of the ventilation hole by gravitation. As a result, the water resistance of the blood pressure measurement device is improved.

In the blood pressure measurement device according to the one aspect described above, there is provided a blood pressure measurement device further including a pump accommodated inside the case and configured to compress air inside the case and feed the air to the cuff structure.

According to this aspect, when the pump draws the air residing inside the case and the case interior becomes negative in pressure, air flows from outside the case to inside the case through the flow path. The water-repellent portion provided on the flow path suppresses the entry of water into the flow path, making it possible to improve the water resistance of the blood pressure measurement device.

In the blood pressure measurement device according to the one aspect described above, there is provided a blood pressure measurement device further including a moisture-permeable waterproof filter provided at the first open end and configured to allow passage of air and to regulate passage of water.

According to this aspect, even in the unlikely event that water enters the ventilation hole, the entry of water into the case can be suppressed by the moisture-permeable waterproof filter, making it possible to improve the water resistance of the blood pressure measurement device.

In the blood pressure measurement device according to the one aspect described above, there is provided a blood pressure measurement device further including a hydrophilic portion provided around the water-repellent portion provided on the outer surface of the at least one of the case or the cuff structure that constitutes the flow path.

According to this aspect, it is possible to retain the water moving toward the flow path in the hydrophilic portion, and retain the water moving from the water-repellent portion in the hydrophilic portion. Thus, the entry of water into the flow path can be suppressed, making it possible to improve the water resistance of the blood pressure measurement device.

In the blood pressure measurement device according to the one aspect described above, there is provided a blood pressure measurement device further including a curler curving to follow a circumferential direction of a site of a living body where the curler is attached, the curler being formed with a first end and a second end spaced apart from each other, the cuff structure being provided on the curler, wherein the flow path is constituted by at least one of the case, the cuff structure, or the curler, and the water-repellent portion is provided on at least one of an inner surface of the flow path or a portion of an outer surface of the at least one of the case, the cuff structure, or the curler that constitutes the flow path, the portion being continuous with the flow path.

According to this aspect, in the blood pressure measurement device having a configuration including the curler, the entry of water from outside the case into the flow path is suppressed by an action of pushing the water about to enter the flow path to outside the flow path, which is exerted by the water-repellent portion provided on the inner surface of the flow path. Further, the water-repellent portion provided on a portion of the outer surface of a member constituting the flow path, the portion being continuous with the flow path, suppresses adherence of the water to the portion. With the entry of water into the flow path being suppressed due to any of these effects, the entry of water into the case is suppressed. As a result, the water resistance of the blood pressure measurement device is improved.

In the blood pressure measurement device according to the one aspect described above, there is provided a blood pressure measurement device further including a hydrophilic portion provided around the water-repellent portion provided on the outer surface of the at least one of the case, the cuff structure, or the curler that constitutes the flow path.

According to this aspect, in the blood pressure measurement device having a configuration including the curler, it is possible to retain the water moving toward the flow path in the hydrophilic portion, and retain the water moving from the water-repellent portion in the hydrophilic portion. Thus, the entry of water into the flow path can be suppressed, making it possible to improve the water resistance of the blood pressure measurement device.

### Advantageous Effects of Invention

The present invention can provide a blood pressure measurement device that can improve waterproof performance.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a perspective view illustrating a configuration of a blood pressure measurement device according to a first embodiment of the present invention.

FIG. 2 is an exploded perspective view illustrating the configuration of the blood pressure measurement device.

FIG. 3 is a perspective view illustrating the configuration of the blood pressure measurement device.

FIG. 4 is a perspective view illustrating a configuration of an outer case of the blood pressure measurement device.

FIG. 5 is a cross-sectional view illustrating the configuration of the outer case.

FIG. 6 is an explanatory diagram illustrating a state in which the blood pressure measurement device is attached to a wrist.

FIG. 7 is a block diagram illustrating the configuration of the blood pressure measurement device.

FIG. 8 is an exploded perspective view illustrating configurations of a curler and a cuff structure of the blood pressure measurement device.

FIG. 9 is a cross-sectional view illustrating the configurations of the curler and the cuff structure of the blood pressure measurement device.

FIG. 10 is a cross-sectional view illustrating the configurations of the curler and the cuff structure of the blood pressure measurement device.

FIG. 11 is a cross-sectional view illustrating a configuration of a back-side cuff of the blood pressure measurement device.

FIG. 12 is a cross-sectional view illustrating the configuration of the back-side cuff of the blood pressure measurement device.

FIG. 13 is a perspective view illustrating the configuration of the curler of the blood pressure measurement device.

FIG. 14 is a plan view illustrating the configuration of the cuff structure of the blood pressure measurement device.

FIG. 15 is a plan view illustrating the configuration of the cuff structure.

FIG. 16 is a plan view illustrating a configuration of a palm-side cuff of the blood pressure measurement device.

FIG. 17 is a cross-sectional view illustrating the configuration of the palm-side cuff.

FIG. 18 is a plan view illustrating a configuration of a sensing cuff of the blood pressure measurement device.

FIG. 19 is a cross-sectional view illustrating the configuration of the sensing cuff of the blood pressure measurement device.

FIG. 20 is a cross-sectional view schematically illustrating the configurations of the curler and the cuff structure of the blood pressure measurement device.

FIG. 21 is a cross-sectional view schematically illustrating a configuration of a power supply unit of the blood pressure measurement device.

FIG. 22 is a flowchart illustrating an example of usage of the blood pressure measurement device.

FIG. 23 is a perspective view illustrating an example in which the blood pressure measurement device is attached to the wrist.

FIG. 24 is a perspective view illustrating an example in which the blood pressure measurement device is attached to the wrist.

FIG. 25 is a perspective view illustrating an example in which the blood pressure measurement device is attached to the wrist.

FIG. 26 is a cross-sectional view schematically illustrating a state in which the blood pressure measurement device is attached to a living body.

FIG. 27 is a cross-sectional view illustrating another configuration of the outer case of the blood pressure measurement device.

### DESCRIPTION OF EMBODIMENTS

### First Embodiment

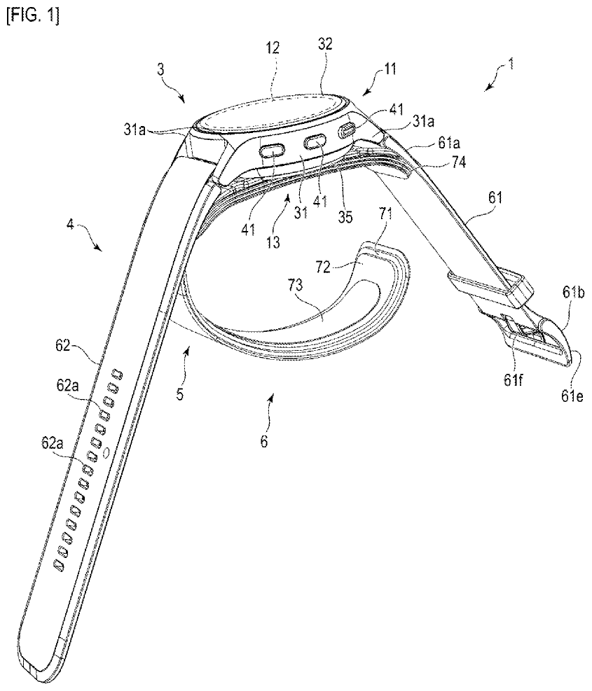

An example of a blood pressure measurement device 1 according to a first embodiment of the present invention will be described below using FIG. 1 to FIG. 25.

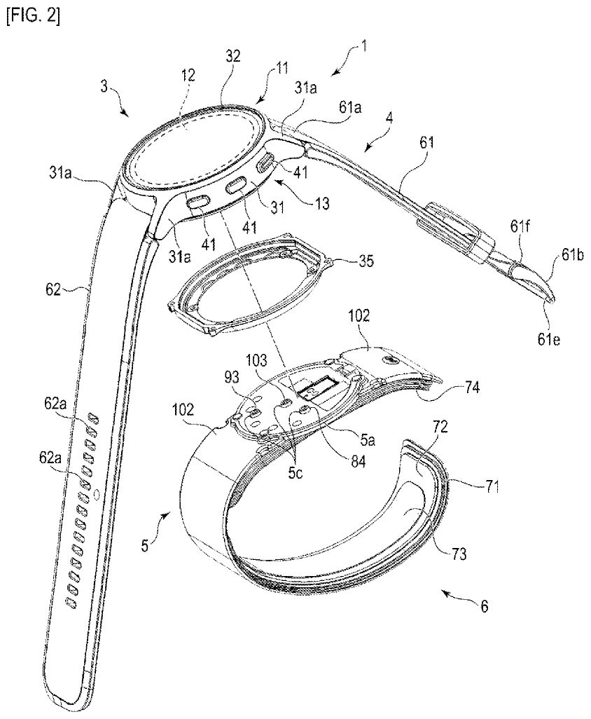

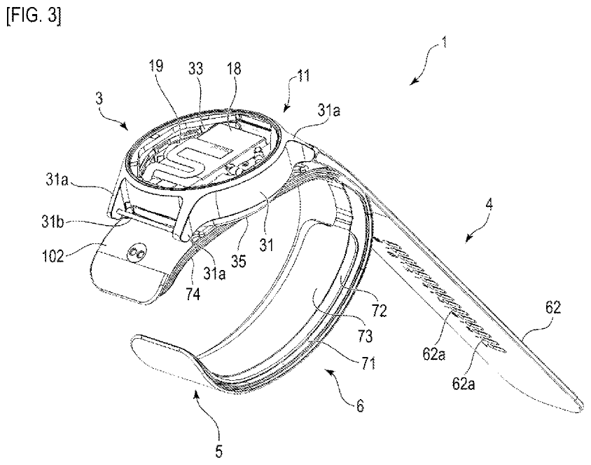

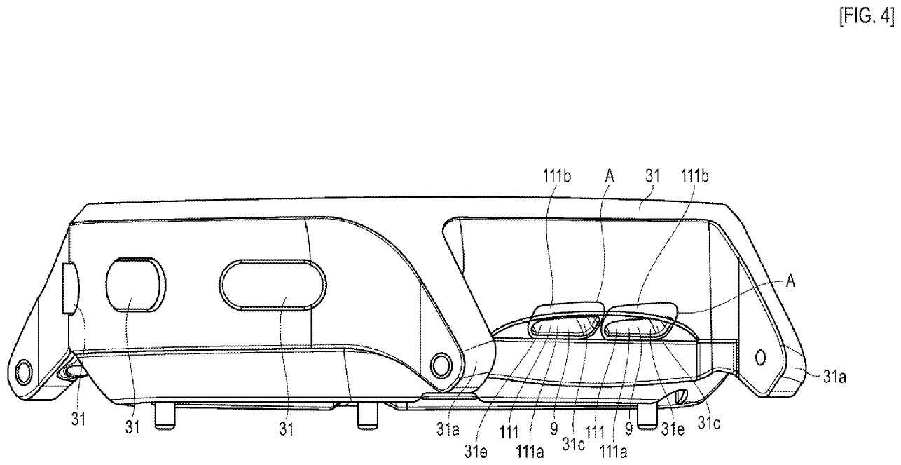

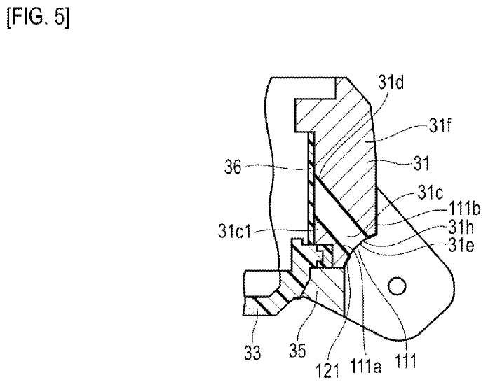

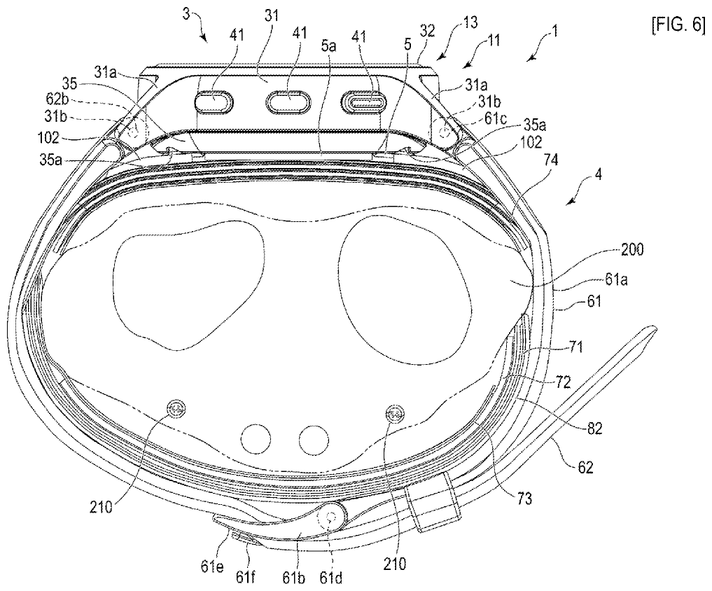

FIG. 1 is a perspective view illustrating a configuration of the blood pressure measurement device 1 according to the first embodiment of the present invention. FIG. 2 is an exploded perspective view illustrating the configuration of the blood pressure measurement device 1. FIG. 3 is a perspective view illustrating the configuration of the blood pressure measurement device 1. FIG. 4 is a perspective view illustrating a configuration of an outer case 31 of the blood pressure measurement device 1. FIG. 5 is a cross-sectional view illustrating the configuration of the outer case 31. FIG. 6 is an explanatory diagram illustrating a state in which the blood pressure measurement device 1 is attached to a wrist 200.

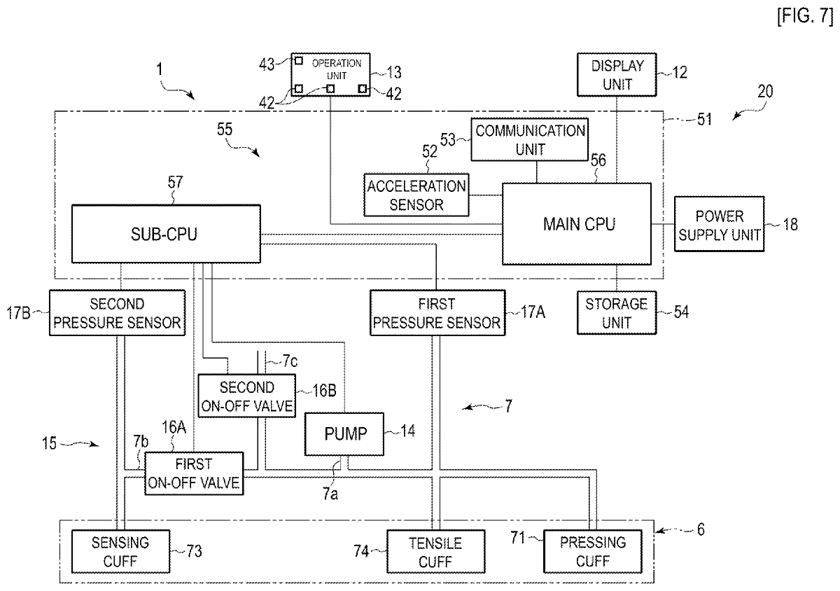

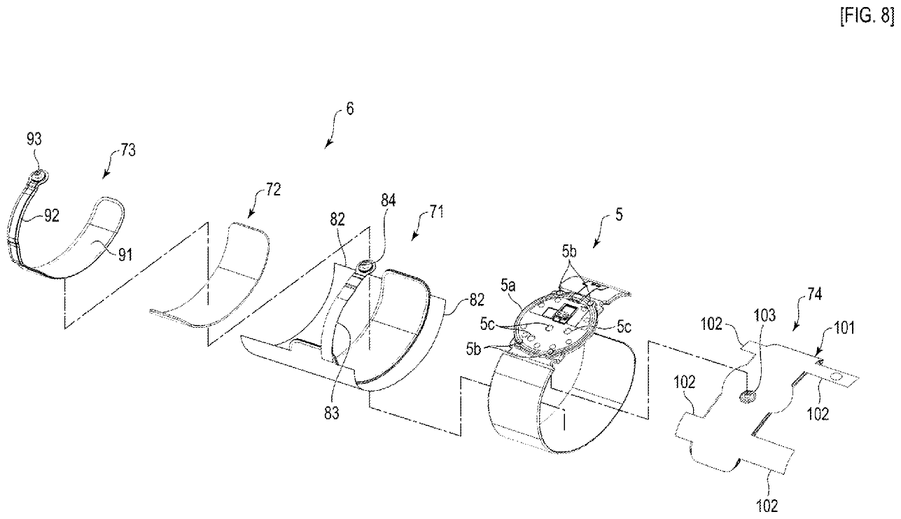

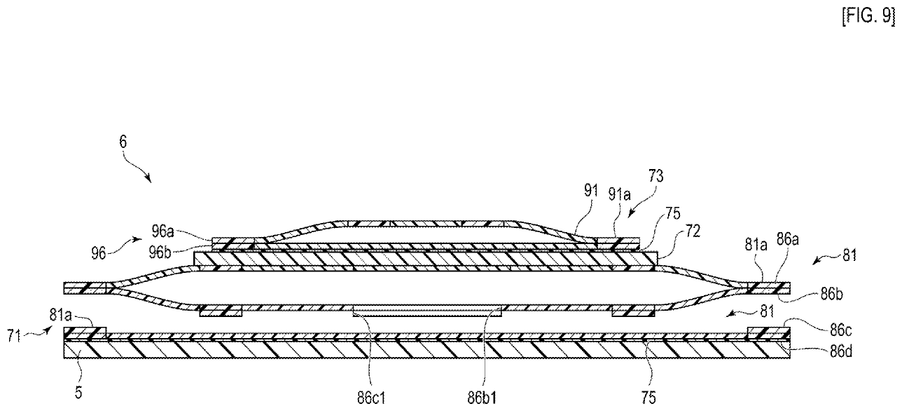

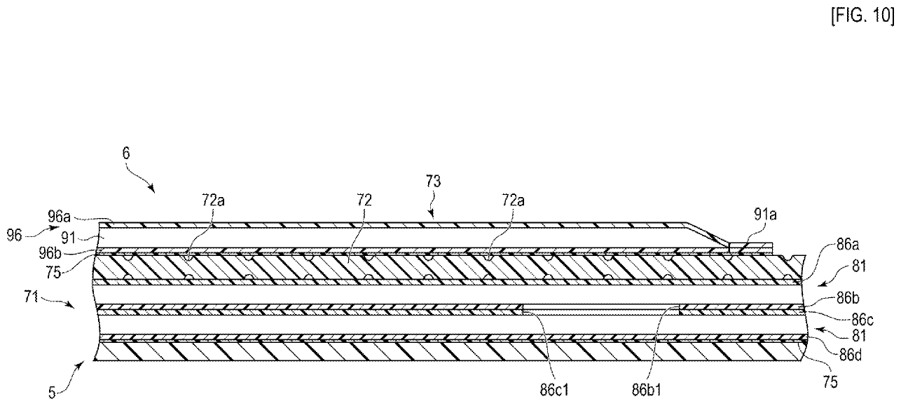

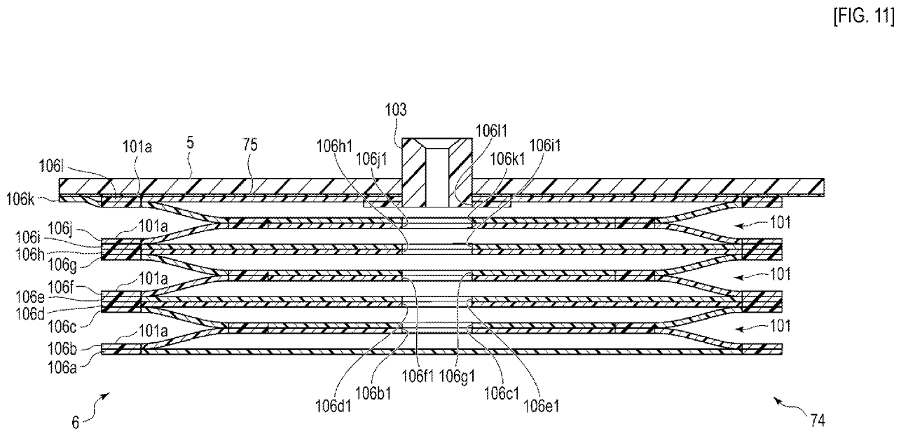

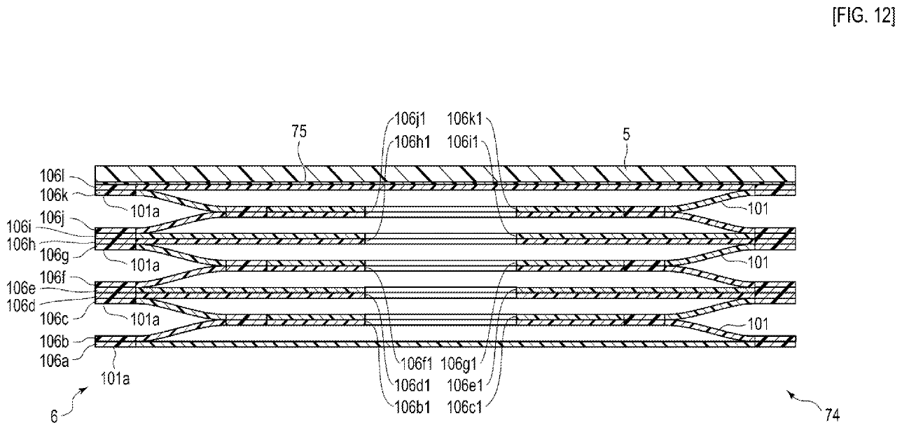

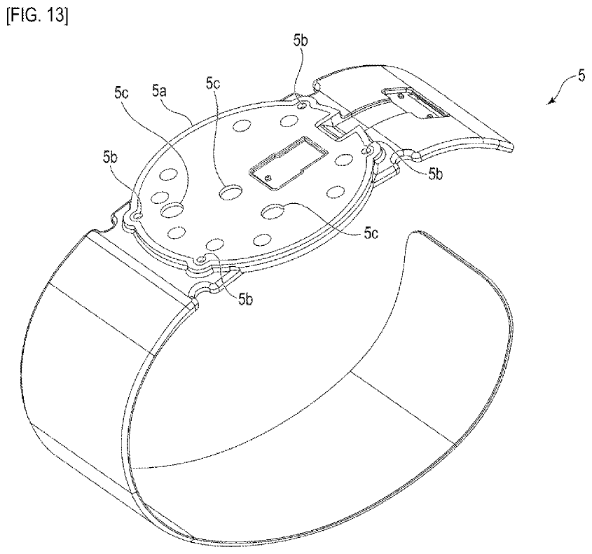

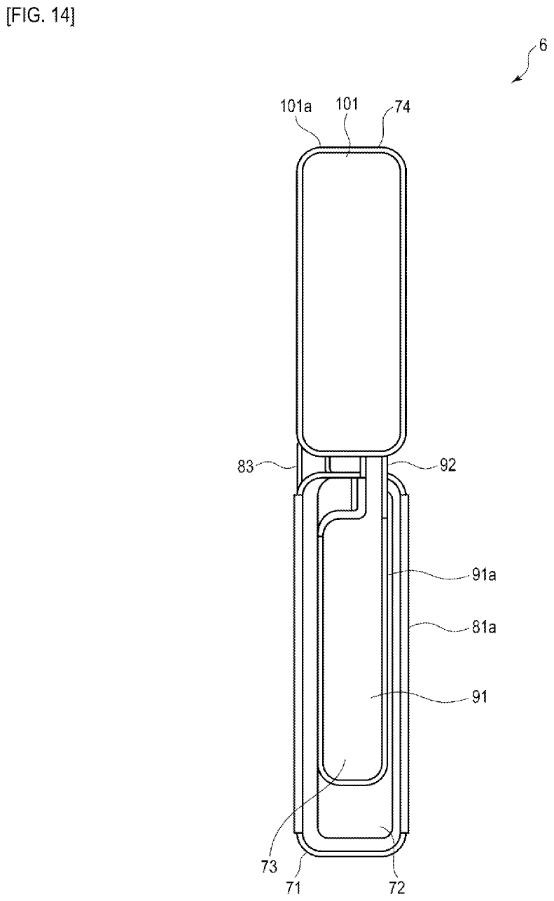

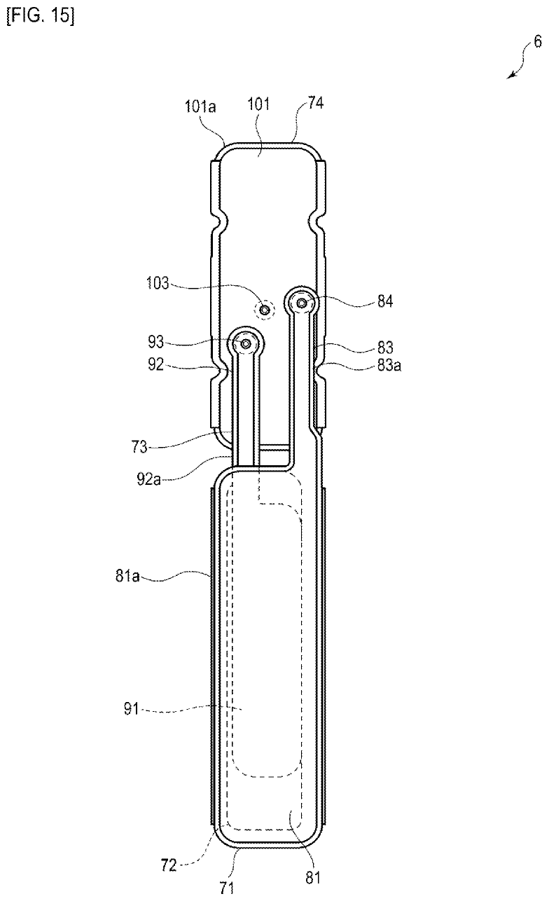

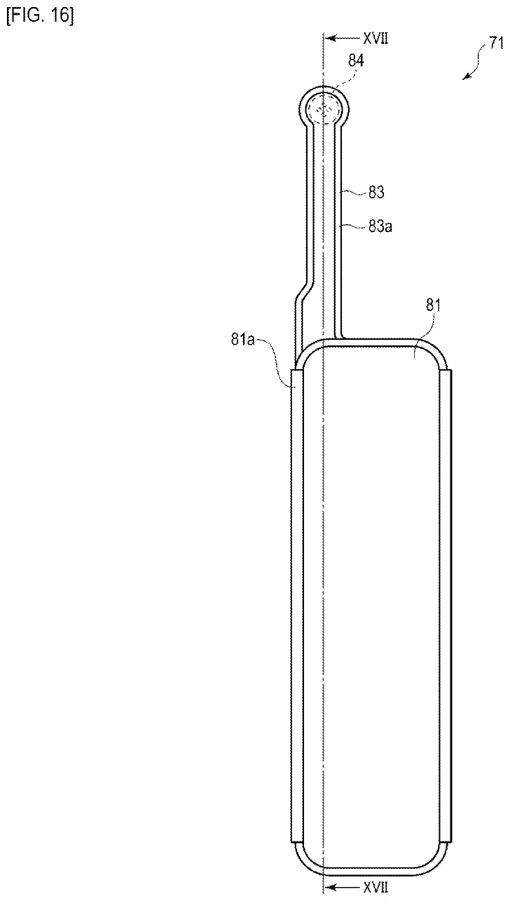

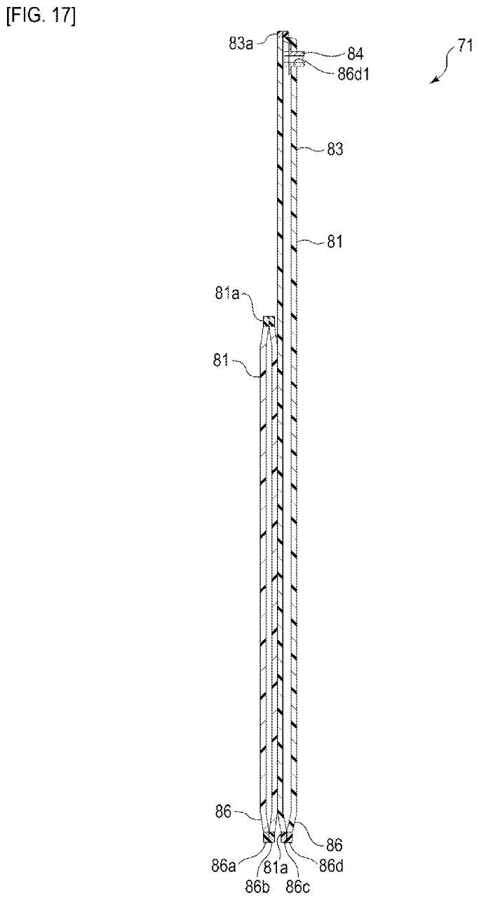

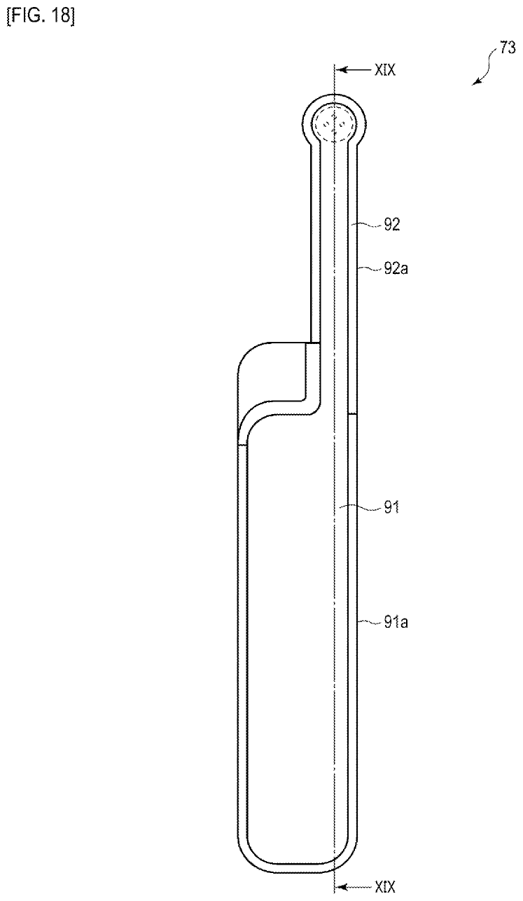

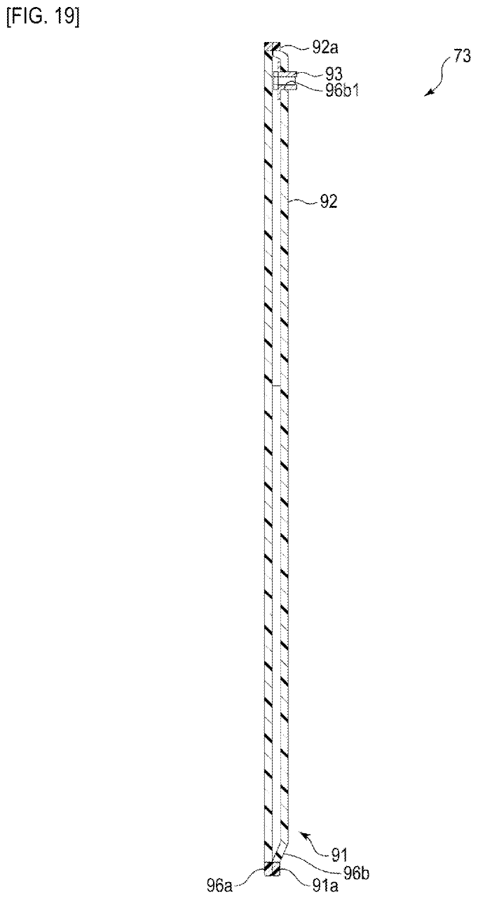

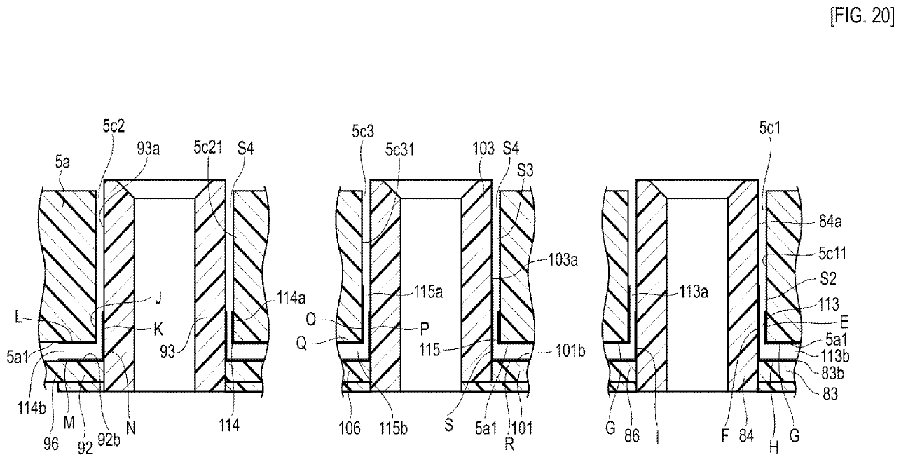

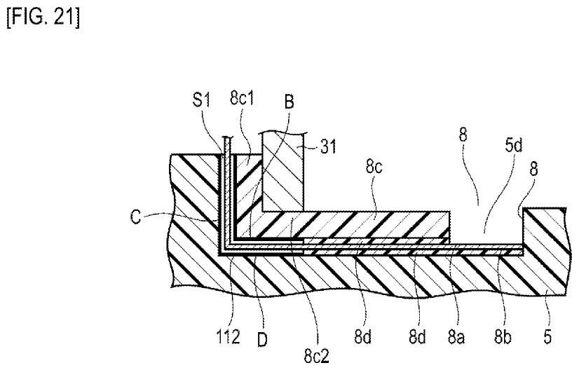

FIG. 7 is a block diagram illustrating the configuration of the blood pressure measurement device 1. FIG. 8 is an exploded perspective view illustrating configurations of a curler 5 and a cuff structure 6 of the blood pressure measurement device 1. FIG. 9 is a cross-sectional view illustrating the configurations of the curler 5 and the cuff structure 6 of the blood pressure measurement device 1. FIG. 10 is a cross-sectional view illustrating the configurations of the curler 5 and the cuff structure 6 of the blood pressure measurement device 1. FIG. 11 is a cross-sectional view illustrating a configuration of a back-side cuff 74 of the blood pressure measurement device 1. FIG. 12 is a cross-sectional view illustrating the configuration of the back-side cuff 74 of the blood pressure measurement device 1. FIG. 13 is a perspective view illustrating the configuration of the curler 5 of the blood pressure measurement device 1. FIG. 14 is a plan view illustrating a configuration of the cuff structure 6 of the blood pressure measurement device 1 on a living body side. FIG. 15 is a plan view illustrating the cuff structure 6 as viewed from an inner circumferential surface side of the curler 5. FIG. 16 is a plan view illustrating a configuration of a palm-side cuff 71 of the blood pressure measurement device 1. FIG. 17 is a cross-sectional view of the configuration of the palm-side cuff 71 taken along line XVII-XVII in FIG. 16. FIG. 18 is a plan view illustrating a configuration of a sensing cuff 73 of the blood pressure measurement device 1. FIG. 19 is a cross-sectional view of the configuration of the sensing cuff 73 of the blood pressure measurement device 1 taken along line XIX-XIX in FIG. 18. FIG. 20 is a cross-sectional view schematically illustrating the configurations of the curler 5 and the cuff structure 6 of the blood pressure measurement device 1. FIG. 21 is a cross-sectional view schematically illustrating a configuration of a power supply unit 18 of the blood pressure measurement device.

The blood pressure measurement device 1 is an electronic blood pressure measurement device attached to a living body. The present embodiment will be described using an electronic blood pressure measurement device having an aspect of a wearable device attached to the wrist 200 of the living body.

As illustrated in FIG. 1 to FIG. 3 and FIG. 7, the blood pressure measurement device 1 includes a device body 3, a belt 4 that fixes the device body 3 to the wrist, the curler 5 disposed between the belt 4 and the wrist, the cuff structure 6 including the palm-side cuff 71, the sensing cuff 73, and the back-side cuff 74, and a fluid circuit 7 fluidly connecting the device body 3 and the cuff structure 6. Furthermore, as illustrated in FIG. 4, FIG. 5, FIG. 20, and FIG. 21, the blood pressure measurement device 1 includes a power supply unit 8 and a water-repellent portion 9.

As illustrated in FIG. 1 to FIG. 7, the device body 3 includes, for example, a case 11, a display unit 12, an operation unit 13, a pump 14, a flow path unit 15, an on-off valve 16, a pressure sensor 17, the power supply unit 18, a vibration motor 19, and a control substrate 20. The device body 3 feeds a fluid to the cuff structure 6 using the pump 14, the on-off valve 16, the pressure sensor 17, the control substrate 20, and the like.

As illustrated in FIG. 1 to FIG. 3, the case 11 includes the outer case 31, a windshield 32 that covers an upper opening of the outer case 31, a base portion 33 provided at a lower portion of an interior of the outer case 31, a back cover 35 covering a lower portion of the outer case 31, and a moisture-permeable waterproof filter 36.

The outer case 31 is formed in a cylindrical shape. The outer case 31 includes pairs of lugs 31a provided at circumferentially symmetrical positions on the outer circumferential surface, and spring rods 31b each provided between corresponding lugs 31 of the two pairs of lugs 31a. As illustrated in FIG. 4 and FIG. 5, an end portion of the outer circumferential surface of the outer case 31 on the back cover 35 side between the pair of lugs 31a is chamfered and configured as a surface inclined with respect to a center line of the outer case 31. This chamfered portion of an outer circumferential surface 31f is referred to as a chamfered portion 31h.

As illustrated in FIG. 4, a hole 31c is formed between each of the two pairs of lugs 31a of the outer case 31. The hole 31c passes through the outer case 31 in a thickness direction. A plurality of the holes 31c, for example, two holes 31c, are formed between the pair of lugs 31a. These two holes 31c are disposed side by side in a circumferential direction of the outer case 31.

As illustrated in FIG. 5, each hole 31c is constituted in a shape that extends linearly on the back cover 35 side from an inner side toward an outer side of the outer case 31. The hole 31c includes a first open end 31d disposed on an inner surface of the outer case 31, and a second open end 31e disposed on an outer surface of the outer case 31.

The first open end 31d is positioned on the windshield 32 side in an axial direction of the outer case 31 with respect to the second open end 31e. The second open end 31e is disposed in the chamfered portion 31 h of the outer case 31 on the back cover 35 side. The hole 31c is constituted in a shape such that an extending direction of the hole 31c is inclined with respect to the center line of the outer case 31, for example. The extending direction of the hole 31c is set in a direction inclined by, for example, 45 degrees with respect to the center line of the outer case 31.

The windshield 32 is, for example, a circular glass plate.

The base portion 33 holds the display unit 12, the operation unit 13, the pump 14, the on-off valve 16, the pressure sensor 17, the power supply unit 18, the vibration motor 19, and the control substrate 20. Additionally, the base portion 33 constitutes, for example, a portion of the flow path unit 15 that makes the pump 14 and the cuff structure 6 fluidly continuous.

The back cover 35 is constituted in an annular shape in which a central side is open. The back cover 35 covers an outer circumferential edge side of an end portion of the outer case 31 on the living body side. Such a back cover 35 is integrally combined with the curler 5 so that the central opening is covered by the curler 5, and, together with the curler 5, constitutes a back lid that covers the end portion of the outer case 31 on the living body side. The back cover 35 is fixed to the end portion of the outer case 31 on the living body side or to the base portion 33 using, for example, four screws 35a.

As illustrated in FIG. 5, the moisture-permeable waterproof filter 36 is provided at the first open end 31d of the hole 31c. The moisture-permeable waterproof filter 36 covers the first open end 31d. The moisture-permeable waterproof filter 36 is a filter having a function of allowing the passage of air and regulating the passage of water. The term “regulating” here refers to limiting an amount of water passage. Alternatively, the moisture-permeable waterproof filter 36 preferably has the ability to prevent the passage of water.

The display unit 12 is disposed above the base portion 33 of the outer case 31 and directly below the windshield 32. As illustrated in FIG. 7, the display unit 12 is electrically connected to the control substrate 20. The display unit 12 is, for example, a liquid crystal display or an organic electroluminescence display. The display unit 12 displays various types of information including the date and time and measurement results of blood pressure values such as the systolic blood pressure and diastolic blood pressure, heart rate, and the like.

The operation unit 13 is configured to be capable of receiving an instruction input from a user. For example, the operation unit 13 includes a plurality of buttons 41 provided on the case 11, a sensor 42 that detects operation of the buttons 41, and a touch panel 43 provided on the display unit 12 or the windshield 32, as illustrated in FIG. 7. When operated by the user, the operation unit 13 converts an instruction into an electrical signal. The sensor 42 and the touch panel 43 are electrically connected to the control substrate 20 and output electrical signals to the control substrate 20.

As the plurality of buttons 41, for example, three buttons are provided. The buttons 41 are supported by the base portion 33 and protrude from the outer circumferential surface of the outer case 31. The plurality of buttons 41 and a plurality of the sensors 42 are supported by the base portion 33. The touch panel 43 is integrally provided on the windshield 32, for example.

The pump 14 is, for example, a piezoelectric pump. The pump 14 compresses air and feeds the compressed air to the cuff structure 6 through the flow path unit 15. The pump 14 is electrically connected to the control substrate 20.

The flow path unit 15 constitutes a flow path connecting from the pump 14 to the palm-side cuff 71 and the back-side cuff 74 and a flow path connecting from the pump 14 to the sensing cuff 73, as illustrated in FIG. 7. Additionally, the flow path unit 15 constitutes a flow path connecting from the palm-side cuff 71 and the back-side cuff 74 to the atmosphere, and a flow path connecting from the sensing cuff 73 to the atmosphere. The flow path unit 15 is a flow path of air constituted by a hollow portion, a groove, a tube, and the like provided in the base portion 33 and the like.

The on-off valve 16 opens and closes a portion of the flow path unit 15. A plurality of the on-off valves 16 are provided, for example, as illustrated in FIG. 7, and selectively open and close the flow path connecting from the pump 14 to the palm-side cuff 71 and the back-side cuff 74, the flow path connecting from the pump 14 to the sensing cuff 73, the flow path connecting from the palm-side cuff 71 and the back-side cuff 74 to the atmosphere, and the flow path connecting from the sensing cuff 73 to the atmosphere, by the combination of opening and closing of each of the on-off valves 16. For example, two on-off valves 16 are used.

The pressure sensor 17 detects the pressures in the palm-side cuff 71, the sensing cuff 73 and the back-side cuff 74. The pressure sensor 17 is electrically connected to the control substrate 20. The pressure sensor 17 converts a detected pressure into an electrical signal, and outputs the electrical signal to the control substrate 20. The pressure sensor 17 is provided, for example, in each of the flow path connecting from the pump 14 to the palm-side cuff 71 and the back-side cuff 74 and the flow path connecting from the pump 14 to the sensing cuff 73, as illustrated in FIG. 7. These flow paths are continuous to the palm-side cuff 71, the sensing cuff 73, and the back-side cuff 74, and thus the pressure in these flow paths are equal to the pressure in the internal space of the palm-side cuff 71, the sensing cuff 73, and the back-side cuff 74.

The power supply unit 18 is, for example, a secondary battery such as a lithium ion battery. The power supply unit 18 is electrically connected to the control substrate 20 as illustrated in FIG. 7. The power supply unit 18 supplies power to the control substrate 20.

As illustrated in FIG. 7, the control substrate 20 includes, for example, a substrate 51, an acceleration sensor 52, a communication unit 53, a storage unit 54, and a control unit 55. The control substrate 20 is constituted by the acceleration sensor 52, the communication unit 53, the storage unit 54, and the control unit 55 that are mounted on the substrate 51.

The substrate 51 is fixed to the base portion 33 of the case 11 using screws or the like.

The acceleration sensor 52 is, for example, a 3-axis acceleration sensor. The acceleration sensor 52 outputs, to the control unit 55, an acceleration signal representing acceleration of the device body 3 in three directions orthogonal to one another. For example, the acceleration sensor 52 is used to measure, from the detected acceleration, the amount of activity of the living body to which the blood pressure measurement device 1 is attached.

The communication unit 53 is configured to be able to transmit and receive information to and from an external device wirelessly or by wire. For example, the communication unit 53 transmits, to an external device via a network, information controlled by the control unit 55, and information of a measured blood pressure value, a pulse, and the like, receives a program or the like for software update from an external device via a network and sends the program or the like to the control unit 55.

In the present embodiment, the network is, for example, the Internet, but is not limited to this. The network may be a network such as a Local Area Network (LAN) provided in a hospital or may be direct communication with an external device using a cable or the like including a terminal of a predetermined standard such as a USB. Thus, the communication unit 53 may be configured to include a plurality of wireless antennas, micro-USB connectors, and the like.

The storage unit 54 pre-stores program data for controlling the overall blood pressure measurement device 1 and the fluid circuit 7, setting data for setting various functions of the blood pressure measurement device 1, calculation data for calculating a blood pressure value and a pulse from pressure measured by the pressure sensors 17, and the like. Additionally, the storage unit 54 stores information such as a measured blood pressure value and a measured pulse.

The control unit 55 is constituted by one or more CPUs, and controls operation of the overall blood pressure measurement device 1 and operation of the fluid circuit 7. The control unit 55 is electrically connected to and supplies power to the display unit 12, the operation unit 13, the pump 14, the on-off valves 16, and the pressure sensors 17. Additionally, the control unit 55 controls operation of the display unit 12, the pump 14, and the on-off valves 16, based on electrical signals output by the operation unit 13 and the pressure sensors 17.

For example, as illustrated in FIG. 7, the control unit 55 includes a main Central Processing Unit (CPU) 56 that controls operation of the overall blood pressure measurement device 1, and a sub-CPU 57 that controls operation of the fluid circuit 7. For example, the main CPU 56 obtains measurement results such as blood pressure values, for example, the systolic blood pressure and the diastolic blood pressure, and the heart rate, from electrical signals output by the pressure sensors 17, and outputs an image signal corresponding to the measurement results to the display unit 12.

For example, the sub-CPU 57 drives the pump 14 and the on-off valves 16 to feed compressed air to the palm-side cuff 71 and the sensing cuff 73 when an instruction to measure the blood pressure is input from the operation unit 13. In addition, the sub-CPU 57 controls driving and stopping of the pump 14 and opening and closing of the on-off valves 16 based on electrical signals output by the pressure sensors 17. The sub-CPU 57 controls the pump 14 and the on-off valves 16 to selectively feed compressed air to the palm-side cuff 71 and the sensing cuff 73 and selectively depressurize the palm-side cuff 71 and the sensing cuff 73.

As illustrated in FIG. 1 to FIG. 6, the belt 4 includes a first belt 61 provided at a first pair of lugs 31a and a first spring rod 31b, and a second belt 62 provided at a second pair of lugs 31a and a second spring rod 31b. The belt 4 is wrapped around the wrist 200 with the curler 5 interposed therebetween.

The first belt 61 is referred to as a so-called a parent and is constituted in a band-like shape that can be coupled with the second belt 62. The first belt 61 includes a belt portion 61a and a buckle 61b, as illustrated in FIG. 1 and FIG. 2. The belt portion 61a is formed in a band-like shape. The belt portion 61a is formed from an elastically deformable resin material. Further, the belt portion 61a includes therein a sheet-like insert member that is flexible and suppresses expansion and contraction of the belt portion 61a in a longitudinal direction. The belt portion 61a includes a first hole portion 61c formed at a first end portion and extending orthogonally to the longitudinal direction of the belt portion 61a, and a second hole portion 61d formed at a second end portion and extending orthogonally to the longitudinal direction of the first belt 61.

As illustrated in FIG. 1 and FIG. 2, the first hole portion 61c is provided at the end portion of the belt portion 61a. The first hole portion 61c has an inner diameter that allows the spring rod 31b to be inserted into the first hole portion 61c and first belt 61 to rotate with respect to the spring rod 31b. In other words, the first belt 61 is rotatably held by the outer case 31 by disposing the first hole portion 61c between the pair of lugs 31a and around the spring rod 31b.

As illustrated in FIG. 1 and FIG. 2, the second hole portion 61d is provided at a distal end of the belt portion 61a. The buckle 61b is attached to the second hole portion 61d.

As illustrated in FIG. 1 and FIG. 2, the buckle 61b includes a frame body 61e having a rectangular frame shape and a prong 61f rotatably attached to the frame body 61e. A side of the frame body 61e to which the prong 61f is attached is inserted into the second hole portion 61d, and the frame body 61e is attached rotatably with respect to the belt portion 61a.

The second belt 62 is referred to as a so-called pointed end, and is constituted in a band-like shape having a width that allows the second belt 62 to be inserted into the frame body 61e. The second belt 62 is formed from an elastically deformable resin material. Further, the second belt 62 includes a sheet-like insert member that is flexible and suppresses expansion and contraction of the second belt 62 in the longitudinal direction.

Further, the second belt 62, as illustrated in FIG. 1 to FIG. 3, includes a plurality of small holes 62a into which the prong 61f is inserted. Additionally, the second belt 62 includes a third hole portion 62b provided at a first end portion of the second belt 62 and extending orthogonally to the longitudinal direction of the second belt 62. The third hole portion 62b has an inner diameter that allows the spring rod 31b to be inserted into the third hole portion 62b and the second belt 62 to rotate with respect to the spring rod 31b. In other words, the second belt 62 is rotatably held by the outer case 31 by disposing the third hole portion 62b between the pair of lugs 31a and around the spring rod 31b.

The second belt 62 of the belt 4 as described above is inserted into the frame body 61e, and the prong 61f is inserted into the small hole 62a. Thus, the first belt 61 and the second belt 62 are integrally connected together, and the belt 4, together with the outer case 31, has an annular shape following the circumferential direction of the wrist 200. With the belt 4 formed in an annular shape following the circumferential direction of the wrist 200, the belt 4 presses and elastically deforms the curler 5, causing the curler 5 to follow the circumferential direction of the wrist of the wearer of the blood pressure measurement device 1.

As illustrated in FIG. 1 to FIG. 6, the curler 5 is constituted in a band-like shape that curves to follow the circumferential direction of the wrist. The curler 5 is formed with a first end and a second end spaced apart from each other. For example, a first end-side outer surface of the curler 5 is fixed to the back cover 35 of the device body 3. The curler 5 is disposed in a position in which the first end and the second end protrude relative to the back cover 35. Furthermore, the first end and the second end of the curler 5 are located adjacent to each other at a predetermined distance from each other. The curler 5 is formed of a resin material, for example. As a specific example, the curler 5 is formed of polypropylene to a thickness of approximately 1 mm.

As a specific example, as illustrated in FIG. 6, the curler 5 is constituted in a band-like shape that curves to follow the circumferential direction of the wrist, and includes a disc-shaped cover portion 5a that, together with the back cover 35, constitute a back lid at a position on the end side facing a hand back side of the wrist 200. In the curler 5, for example, the cover portion 5a and areas adjacent thereto are formed in flat plate shapes, and the first end side and the second end side of the curler 5 that are more distal than the cover portion 5a are formed to curve at a predetermined curvature.

Further, as illustrated in FIG. 13, the curler 5 is formed in a shape in which the second end is positioned on the inner circumferential surface side of the first end side when the first end and the second end are in close proximity. As a specific example, a width of the curler 5 in a width direction of the wrist 200 is set greater on the hand back side of the wrist 200 than on the palm side of the wrist 200. Then, in the curler 5, a radius of curvature of the first end on the hand back side of the wrist 200 is set greater than a radius of curvature of the second end on the palm side of the wrist 200. According to such a configuration, when both end sides of the curler 5 come into contact with each other, the second end of the curler 5 is disposed further inward of the curler 5 than the first end.

The cover portion 5a includes an insert member for reinforcement. The cover portion 5a includes a screw hole 5b to which the back cover 35 is attached using the screw 35a or the like. Further, the cover portion 5a includes a hole portion 5c for connecting the cuff structure 6 to the device body 3. In the present embodiment, three hole portions 5c are provided in the cover portion 5a, each formed to a diameter that allows connection portions 84, 93, and 103 described later of the palm-side cuff 71, the sensing cuff 73, and the back-side cuff 74 to be inserted. Here, the hole portion 5c into which the connection portion 84 of the palm-side cuff 71 is inserted is referred to as a first hole portion 5c1. The hole portion 5c into which the connection portion 93 of the sensing cuff 73 is inserted is referred to as a second hole portion 5c2. The hole portion 5c into which the connection portion 103 of the back-side cuff 74 is inserted is referred to as a third hole portion 5c3. The cover portion 5a is secured to the living body side of outer case 31 via the fixed back cover 35.

Such a curler 5 is fixed to the outer case 31 with both the first end and the second end facing the second belt 62 of the belt 4. Further, at least at a position facing the palm side of the wrist 200, the curler 5 curves to follow the circumferential direction on the palm side of the wrist 200, thereby holding the cuff structure 6 facing the palm side of the wrist 200 in a state of being curved to follow the shape of the palm side of the wrist 200.

Further, the curler 5 has a hardness with flexibility and shape retainability. Here, “flexibility” means that the shape of the curler 5 deforms in a radial direction when an external force of the belt 4 is applied to the curler 5. For example, “flexibility” means that the shape of the curler 5 deforms in a side view when the curler 5 is pressed by the belt 4 such that the curler 5 approaches the wrist, is along the shape of the wrist, or follows the shape of the wrist. Furthermore, “shape retainability” refers to the ability of the curler 5 to maintain a pre-formed shape when no external force is applied to the curler 5. For example, “shape retainability” in the present embodiment means that the shape of the curler 5 curving along the circumferential direction of the wrist can be maintained.

The cuff structure 6 is disposed on the inner circumferential surface of the curler 5, and is held along the shape of the inner circumferential surface of the curler 5. As a specific example, the palm-side cuff 71 and the back-side cuff 74 are disposed on the inner circumferential surface of the curler 5, and the cuff structure 6 is fixed to the curler 5 by a joining layer 75 provided between the curler 5 and both the palm-side cuff 71 and the back-side cuff 74. In the present embodiment, the joining layer 75 is an adhesive or double-sided tape.

The curler 5 is formed of a resin material. For example, the curler 5 is formed of polypropylene to a thickness of approximately 1 mm.

Further, the power supply unit 8 is provided on an outer surface or an inner surface of the curler 5. As a specific example, as illustrated in FIG. 13, a recess 5d in which the power supply unit 8 is provided is formed in a portion of the outer surface of the cover portion 5a on the first end side and in a portion of the surface of the curler 5 on the device body 3 side.

The recess 5d is formed in a shape extending from a portion of the edge portion of the cover portion 5a between one of the pairs of lugs 31a toward the first end of the cover portion 5a. For example, the recess 5d is configured to be sufficiently deep so that the power supply unit 8 provided therein does not protrude from the outer surface of the curler 5.

As illustrated in FIG. 1 to FIG. 6, FIG. 8, FIG. 14, and FIG. 15, the cuff structure 6 includes the palm-side cuff (cuff) 71, a back plate 72, the sensing cuff 73, and the back-side cuff (cuff) 74. Further, the cuff structure 6 includes the joining layer 75 joining the respective configurations as well as the curler 5 and the cuffs 71 and 74. The cuff structure 6 is fixed to the curler 5. In the cuff structure 6, the palm-side cuff 71, the back plate 72, and the sensing cuff 73 are stacked on one another and disposed on the curler 5, and the back-side cuff 74 is spaced apart from the palm-side cuff 71, the back plate 72, and the sensing cuff 73 and disposed on the curler 5.

As a specific example, as illustrated in FIG. 6, the cuff structure 6 is fixed to the inner circumferential surface of the curler 5 on the palm side of the wrist 200 with the palm-side cuff 71, the back plate 72, and the sensing cuff 73 stacked in this order from the inner circumferential surface of the curler 5 toward the living body side. Further, in the cuff structure 6, the back-side cuff 74 is disposed on the inner circumferential surface of the curler 5 on the hand back side of the wrist 200. Each of the members of the cuff structure 6 is fixed to an adjacent member of the cuff structure 6 in a stacking direction with the joining layer 75.

The palm-side cuff 71 is a so-called pressing cuff. The palm-side cuff 71 is fluidly connected to the pump 14 through the flow path unit 15. The palm-side cuff 71 is inflated to press the back plate 72 and the sensing cuff 73 toward the living body side. The palm-side cuff 71, as illustrated in FIG. 8 to FIG. 10, includes air bags 81 including a plurality of layers, for example, two layers, a tube 83 in communication with the air bags 81, and a connection portion 84 provided at a distal end of the tube 83. Such a palm-side cuff 71 is formed by integrally welding a plurality of sheet members 86.

Here, the air bags 81 are bag-like structures, and in the present embodiment, the blood pressure measurement device 1 is configured to use air with the pump 14, and thus the present embodiment will be described using the air bags. However, in a case where a fluid other than air is used, the bag-like structures may be fluid bags such as liquid bags. The plurality of air bags 81 are stacked and are in fluid communication with one another in the stacking direction.

Each of the air bags 81 is formed in a rectangular bag shape elongated in one direction. Further, a width of the air bag 81 in a lateral direction is set to be the same as a width of the curler 5 in the lateral direction. The air bag 81 is formed by, for example, combining two sheet members 86 and thermally welding the sheet members 86 in a rectangular frame shape elongated in one direction, as illustrated by a welded portion 81a in FIG. 9, FIG. 10, and FIG. 14 to FIG. 17. Further, the two-layer air bag 81 is formed by thermally welding and thus integrally combining two air bags 81, or by welding, to each other, the sheet members 86 of adjacent air bags 81 facing each other and then welding the air bags 81. The two-layer air bag 81 is fluidly continuous via openings provided in the sheet members 86 facing each other.

The tube 83 is integrally provided to one of the air bags 81, for example, to a portion of one longitudinal edge portion of the air bag 81 adjacent to the curler 5, as illustrated in FIG. 8 and FIG. 14 to FIG. 17. As a specific example, the tube 83 is provided at an end portion of the air bag 81 near the device body 3. Further, the tube 83 is formed in a shape elongated in one direction and having a width smaller than the width of the air bag 81 in the lateral direction, and a distal end of the tube 83 is formed in a circular shape. The tube 83 includes the connection portion 84 at the distal end. The tube 83 is connected to the flow path unit 15 via the connection portion 84 and constitutes a flow path between the device body 3 and the air bag 81.

With the connection portion 84 disposed on two sheet members 86, a portion of the sheet members 86 adjacent to a region of the sheet members 86 constituting the air bag 81 is thermally welded in a frame shape elongated in one direction, so that the tube 83 is formed.

Note that the air bag 81 provided with the tube 83 is configured so that a portion of the welded portion 81a for welding the two sheet members 86 in a rectangular frame shape is not welded and is continuous with a welded portion 83a constituting the tube 83, thereby making the air bag 81 and the tube 83 fluidly continuous.

The connection portion 84 is, for example, a nipple. The connection portion 84 is provided at the distal end of the tube 83. A distal end of the connection portion 84 is exposed from the sheet member 86 facing the curler 5, which is one of the two sheet members 86 constituting the tube 83. As illustrated in FIG. 20, the connection portion 84 is inserted into the first hole portion 5c1 of the cover portion 5a of the curler 5. The connection portion 84 is constituted in a shape having a diameter equal to or slightly smaller than the diameter of the first hole portion 5c1.

As a specific example, as illustrated in FIG. 9 and FIG. 10, the palm-side cuff 71 includes a first sheet member 86a, a second sheet member 86b, a third sheet member 86c, and a fourth sheet member 86d in this order from the living body side. The second sheet member 86b constitutes a first-layer air bag 81 along with the first sheet member 86a. The third sheet member 86c is integrally joined to the second sheet member 86b. The fourth sheet member 86d constitutes a second-layer air bag 81 and the tube 83 along with the third sheet member 86c. Note that the palm-side cuff 71 is integrally formed by joining the sheet members 86 adjacent to each other by thermal welding.

The first sheet member 86a and the second sheet member 86b are constituted in the same rectangular shape as the air bag 81, and edge portions of four sides thereof are welded to form the air bag 81. The second sheet member 86b and the third sheet member 86c are disposed facing each other, and include a plurality of openings 86b1 and 86c1 through which the two air bags 81 are fluidly continuous. Further, a periphery of the plurality of openings 86b1 and 86c1 is thermally welded in a four-sided frame shape smaller than that of the four sides by which the air bag 81 is welded, thereby the second sheet member 86b and the third sheet member 86c are integrally joined.

The third sheet member 86c is constituted in a shape capable of constituting the air bag 81 and the tube 83, for example. The fourth sheet member 86d is constituted in a shape capable of constituting the air bag 81 and the tube 83, for example. Further, the fourth sheet member 86d includes a hole portion 86d1 into which the distal end of the connection portion 84 can be inserted, for example.

The third sheet member 86c and the fourth sheet member 86d are disposed facing each other, thermally welded along the edge shapes of the air bag 81 and the tube 83 so that the air bag 81 and the tube 83 are fluidly continuous, and cut into predetermined shapes, thereby constituting the air bag 81 and the tube 83.

In the fourth sheet member 86d, the connection portion 84 is disposed in the hole portion 86d1, and a periphery of the hole portion 86d1 is thermally welded to the connection portion 84. Furthermore, the fourth sheet member 86d is joined to the inner circumferential surface of the curler 5 via the joining layer 75.

The back plate 72, as illustrated in FIG. 9 and FIG. 10, is applied to an outer surface of the first sheet member 86a of the palm-side cuff 71 with the joining layer 75. The back plate 72 is formed in a plate shape using a resin material. The back plate 72 is made of polypropylene, for example, and is formed in a plate shape having a thickness of approximately 1 mm. The back plate 72 has shape followability.

Here, “shape followability” refers to a function in which the backplate 72 can be deformed in such a manner as to follow the shape of a contacted portion of the wrist 200 to be disposed. This contacted portion of the wrist 200 refers to a region of the wrist 200 that faces the back plate 72. This contact includes both direct contact and indirect contact via the sensing cuff 73.

For example, as illustrated in FIG. 10, the back plate 72 includes a plurality of grooves 72a formed in both main surfaces of the back plate 72 and extending in a direction orthogonal to the longitudinal direction. As illustrated in FIG. 10, the plurality of grooves 72a are provided in both main surfaces of the back plate 72. The plurality of grooves 72a provided in one of the main surfaces face the corresponding grooves 72a provided in the other main surface in the thickness direction of the back plate 72. Additionally, the plurality of grooves 72a are disposed at equal intervals in the longitudinal direction of the back plate 72.

In the back plate 72, portions including the plurality of grooves 72a are thinner than portions including no grooves 72a and thus the portions including the plurality of grooves 72a are easily deformed. Accordingly, the back plate 72 is deformed in such a manner as to follow the shape of the wrist 200, and has shape followability of extending in the circumferential direction of the wrist. The back plate 72 is formed such that the length of the back plate 72 is sufficient to cover the palm side of the wrist 200. The back plate 72 transfers the pressing force from the palm-side cuff 71 to the back plate 72 side main surface of the sensing cuff 73 in a state in which the back plate 72 is extending along the shape of the wrist 200.

The sensing cuff 73 is fluidly connected to the pump 14 via the flow path unit 15. The sensing cuff 73 is fixed to the living body side main surface of the back plate 72. The sensing cuff 73 is in direct contact with a region of the wrist 200 where an artery 210 resides, as illustrated in FIG. 6 and FIG. 9. Examples of the artery 210 used herein include a radial artery and an ulnar artery. The sensing cuff 73 is formed in the same shape as that of the back plate 72 or a shape that is smaller than that of the back plate 72, in the longitudinal direction and the width direction of the back plate 72. The sensing cuff 73 is inflated to compress a palm-side region of the wrist 200 in which the artery 210 resides. The sensing cuff 73 is pressed by the inflated palm-side cuff 71 toward the living body side with the back plate 72 in between.

As a specific example, the sensing cuff 73 includes, as illustrated in FIG. 9, FIG. 10, FIG. 18, and FIG. 19, one air bag 91, a tube 92 that communicates with the air bag 91, and the connection portion 93 provided at a distal end of the tube 92. One main surface of the air bag 91 of the sensing cuff 73 is fixed to the back plate 72. For example, the sensing cuff 73 is joined to the living body side main surface of the back plate 72 by the joining layer 75. Such a sensing cuff 73 is formed by integrally welding two sheet members 96.

Here, the air bag 91 is a bag-like structure, and in the present embodiment, the blood pressure measurement device 1 is configured to use air with the pump 14, and thus the present embodiment will be described using the air bag. However, in a case where a fluid other than air is used, the bag-like structure may be a liquid bag and the like.

The air bag 91 is constituted in a rectangular shape elongated in one direction. The air bag 91 is formed by, for example, combining two sheet members 96 elongated in one direction and thermally welding the sheet members 96 in a rectangular frame shape elongated in one direction, as illustrated by a welded portion 91a in FIG. 9, FIG. 10, FIG. 14, FIG. 18, and FIG. 19.

The tube 92 is integrally provided at a portion of one longitudinal edge portion of the air bag 91. As a specific example, the tube 92 is provided at an end portion of the air bag 91 near the device body 3. Further, the tube 92 is formed in a shape elongated in one direction with a width smaller than a width of the air bag 91 in the lateral direction, and the distal end of the tube 92 is formed in a circular shape. The tube 92 includes the connection portion 93 at the distal end. The tube 92 is connected to the flow path unit 15 via the connection portion 93 and constitutes a flow path between the device body 3 and the air bag 91.

With the connection portion 93 disposed on two sheet members 96, a portion of the sheet members 96 adjacent to a region of the sheet members 96 constituting the air bag 91 is thermally welded in a frame shape elongated in one direction, so that the tube 92 is formed. Note that the air bag 91 is configured so that a portion of the welded portion 91a for welding the two sheet members 96 in a rectangular frame shape is not welded and is continuous with a welded portion 92a constituting the tube 92, thereby making the air bag 91 and the tube 92 fluidly continuous.

The connection portion 93 is, for example, a nipple. The connection portion 93 is provided at the distal end of the tube 92. Further, the distal end of the connection portion 93 is exposed to the outside from the sheet member 96 facing the curler 5 and the back plate 72, which is one of the two sheet members 96 constituting the tube 92.

The connection portion 93 is connected to the flow path unit 15. As illustrated in FIG. 20, the connection portion 93 is constituted in a tubular shape protruding from the tube 92. The connection portion 93 is, for example, a nipple. The connection portion 93 is inserted into the second hole portion 5c2 of the cover portion 5a of the curler 5. The connection portion 93 is constituted in a shape having a diameter equal to or slightly smaller than the diameter of the second hole portion 5c2.

As a specific example, the sensing cuff 73 includes a fifth sheet member 96a and a sixth sheet member 96b in this order from the living body side as illustrated in FIG. 9 and FIG. 10. Note that the sensing cuff 73 is formed by joining the sheet members 96 adjacent to each other by thermal welding.

For example, the fifth sheet member 96a and the sixth sheet member 96b are constituted in a shape capable of constituting the air bag 81 and the tube 83. The fifth sheet member 96a and the sixth sheet member 96b are disposed facing each other, thermally welded along the edge shapes of the air bag 91 and the tube 92 so that the air bag 91 and the tube 92 are fluidly continuous, and cut into predetermined shapes, thereby constituting the air bag 91 and the tube 92.

Further, the sixth sheet member 96b includes a hole portion 96b1 into which the distal end of the connection portion 93 can be inserted, for example. In the sixth sheet member 96b, the connection portion 93 is disposed in the hole portion 96b1, and a periphery of the hole portion 96b1 is thermally welded to the connection portion 93. The sixth sheet member 96b is joined to an inner circumferential surface of the back plate 72 via the joining layer 75.

The back-side cuff 74 is a so-called tensile cuff. The back-side cuff 74 is fluidly connected to the pump 14 through the flow path unit 15. The back-side cuff 74 is inflated to press the curler 5 such that the curler 5 is spaced apart from the wrist 200, pulling the belt 4 and the curler 5 toward the hand back side of the wrist 200. The back-side cuff 74 includes air bags 101 including a plurality of layers, for example, six layers, and the connection portion 103 provided at the air bag 101 facing the curler 5. Such a back-side cuff 74 is formed by integrally welding a plurality of the sheet members 106.

Additionally, the back-side cuff 74 is configured such that the thickness of the back-side cuff 74 in an inflating direction during inflation is larger than the thickness of the palm-side cuff 71 in the inflating direction during inflation and than the thickness of the sensing cuff 73 in the inflating direction during inflation. In the present embodiment, the inflating direction is the direction in which the curler 5 and the wrist 200 face each other. Specifically, the air bags 101 of the back-side cuff 74 include more layers than the air bags 81 of the palm-side cuff 71 and the air bag 91 of the sensing cuff 73, and are thicker than the palm-side cuff 71 and the sensing cuff 73 when the air bags 101 are inflated from the curler 5 toward the wrist 200.

Here, the air bag 101 is a bag-like structure, and in the present embodiment, the blood pressure measurement device 1 is configured to use air with the pump 14, and thus the present embodiment will be described using the air bag. However, in a case where a fluid other than air is used, the bag-like structure may be a fluid bag such as a liquid bag. A plurality of the air bags 101 are stacked and are in fluid communication in the stacking direction.

Each of the air bags 101 is formed in a rectangular bag shape elongated in one direction. Further, a width in a lateral direction of the air bag 101 is set to be the same as the width in the lateral direction of the curler 5. The air bag 101 is formed by, for example, combining two sheet members 106 and thermally welding the sheet members 106 in a rectangular frame shape elongated in one direction, as illustrated by a welded portion 101a in FIG. 11, FIG. 12, FIG. 14, and FIG. 15. Further, the six-layer air bags 101 are formed by thermally welding and thus integrally combining six air bags 101, or by welding, to each other, the sheet members 106 of the adjacent air bags 101 facing each other and then welding the air bags 101, for example. The six-layer air bags 101 are fluidly continuous via openings provided in the sheet members 106 facing each other.

The connection portion 103 is, for example, a nipple. The connection portion 103 is provided on a center side in the longitudinal direction of the air bag 101 disposed adjacent to the curler 5. A distal end of the connection portion 103 is exposed from the sheet member 106 facing the curler 5, which is one of the two sheet members 106 constituting the air bag 101.

The connection portion 103 is connected to the flow path unit 15. As illustrated in FIG. 20, the connection portion 103 is constituted in a tubular shape protruding from the air bag 101. The connection portion 103 is, for example, a nipple. The connection portion 103 is inserted into the third hole portion 5c3 of the cover portion 5a of the curler 5. The connection portion 103 has a diameter equal to or slightly smaller than the diameter of the third hole portion 5c3.

As a specific example, as illustrated in FIG. 11 and FIG. 12, the back-side cuff 74 includes a seventh sheet member 106a, an eighth sheet member 106b, a ninth sheet member 106c, a tenth sheet member 106d, an eleventh sheet member 106e, a twelfth sheet member 106f, a thirteenth sheet member 106 g, a fourteenth sheet member 106h, a fifteenth sheet member 106i, a sixteenth sheet member 106j, a seventeenth sheet member 106k, and an eighteenth sheet member 106 l in this order from the living body side. Note that the back-side cuff 74 is integrally formed by joining the sheet members 106 adjacent to each other by thermal welding.

The seventh sheet member 106a to the eighteenth sheet member 106 l are each constituted in a rectangular shape similar to that of the air bags 101. Edge portions of four sides of the seventh sheet member 106a are welded to corresponding edge portions of four sides of the eighth sheet member 106b to constitute a first-layer air bag 101. The eighth sheet member 106b and the ninth sheet member 106c are disposed facing each other, and include a plurality of openings 106b1 and 106c1 through which the two air bags 101 are fluidly continuous. Further, a periphery of the plurality of openings 106b1 and 106c1 is thermally welded in a four-sided frame shape smaller than that of the four sides by which the air bag 101 is welded, thereby the eighth sheet member 106b and the ninth sheet member 106c are integrally joined.

Edge portions of four sides of the ninth sheet member 106c are welded to corresponding edge portions of four sides of the tenth sheet member 106d to constitute a second-layer air bag 101.

The tenth sheet member 106d and the eleventh sheet member 106e are, as illustrated in FIG. 11 and FIG. 12, disposed facing each other, and include a plurality of openings 106d1 and 106e1 through which the two air bags 101 are fluidly continuous. Further, a periphery of the plurality of openings 106d1 and 106e1 is thermally welded in a four-sided frame shape smaller than that of the four sides by which the air bag 101 is welded, thereby the tenth sheet member 106d and the eleventh sheet member 106e are integrally joined. Edge portions of four sides of the eleventh sheet member 106e are welded to corresponding edge portions of four sides of the twelfth sheet member 106f to constitute a third-layer air bag 101.

The twelfth sheet member 106f and the thirteenth sheet member 106 g are, as illustrated in FIG. 11 and FIG. 12, disposed facing each other, and include a plurality of openings 106f1 and 106g1 through which the two air bags 101 are fluidly continuous. Further, a periphery of the plurality of openings 106f1 and 106g1 is thermally welded in a four-sided frame shape smaller than that of the four sides by which the air bag 101 is welded, thereby the twelfth sheet member 106f and the thirteenth sheet member 106 g are integrally joined. Edge portions of four sides of the thirteenth sheet member 106 g are welded to corresponding edge portions of four sides of the fourteenth sheet member 106 h to constitute a fourth-layer air bag 101.

The fourteenth sheet member 106 h and the fifteenth sheet member 106i are, as illustrated in FIG. 11 and FIG. 12, disposed facing each other, and include a plurality of openings 106h1 and 106i1 through which the two air bags 101 are fluidly continuous. Further, a periphery of the plurality of openings 106h1 and 106i1 is thermally welded in a four-sided frame shape smaller than that of the four sides by which the air bag 101 is welded, thereby the fourteenth sheet member 106 h and the fifteenth sheet member 106i are integrally joined. Edge portions of four sides of the fifteenth sheet member 106i are welded to corresponding edge portions of four sides of the sixteenth sheet member 106j to constitute a fifth-layer air bag 101.

The sixteenth sheet member 106j and the seventeenth sheet member 106k are, as illustrated in FIG. 11 and FIG. 12, disposed facing each other, and include a plurality of openings 106j1 and 106k1 through which the two air bags 101 are fluidly continuous. Further, the seventeenth sheet member 106k is constituted in a shape capable of constituting the air bag 101, for example. A periphery of the plurality of openings 106j1 and 106k1 is thermally welded in a four-sided frame shape smaller than that of the four sides by which the air bag 101 is welded, thereby the sixteenth sheet member 106j and the seventeenth sheet member 106k are integrally joined. The seventeenth sheet member 106k and the eighteenth sheet member 106 l are thermally welded along the edge shape of the air bag 101 and cut into a predetermined shape to constitute a sixth-layer air bag 101.

Further, the eighteenth sheet member 106 l includes a hole portion 106l1 into which the distal end of the connection portion 103 can be inserted, for example. In the eighteenth sheet member 106l, the connection portion 103 is disposed in the hole portion 106l1, and a periphery of the hole portion 106l1 is thermally welded to the connection portion 103. Further, the eighteenth sheet member 106 l is joined to the inner circumferential surface of the curler 5 via the joining layer 75, and the seventeenth sheet member 106k is joined to an outer circumferential surface of the curler 5 via the joining layer 75.

Additionally, each of the sheet members 86, 96, and 106 forming the palm-side cuff 71, the sensing cuff 73, and the back-side cuff 74 are formed of a thermoplastic resin material. The thermoplastic resin material is a thermoplastic elastomer. Examples of thermoplastic resin material constituting the sheet members 86, 96, and 106 include thermoplastic polyurethane based resin (hereinafter referred to as TPU), polyvinyl chloride resin, ethylene-vinyl acetate resin, thermoplastic polystyrene based resin, thermoplastic polyolefin resin, thermoplastic polyester based resin, and thermoplastic polyamide resin. Of at least the plurality of sheet members 86 and 106 constituting the air bags 81 and 101, at least the sheet members 86 and 106 of the palm-side cuff 71 and the sensing cuff 73 welded to the curler 5 are constituted by a material similar to the material of the curler 5.

For example, the sheet members 86, 96, and 106 are formed using a molding method such as T-die extrusion molding or injection molding. After being molded by each molding method, the sheet members 86, 96, and 106 are sized into predetermined shapes, and the sized individual members are joined by welding or the like to constitute bag-like structures 81, 91, and 101. A high frequency welder or laser welding is used as the welding method.

The fluid circuit 7 is constituted by the case 11, the pump 14, the flow path unit 15, the on-off valves 16, the pressure sensors 17, the palm-side cuff 71, the sensing cuff 73, and the back-side cuff 74. A specific example of the fluid circuit 7 will be described below in which two on-off valves 16 that are used in the fluid circuit 7 are designated as a first on-off valve 16A and a second on-off valve 16B, and two pressure sensors 17 that are used in the fluid circuit 7 are designated as a first pressure sensor 17A and a second pressure sensor 17B.

As illustrated in FIG. 7, the fluid circuit 7 includes, for example, a first flow path 7a connecting the palm-side cuff 71 and the back-side cuff 74 from the pump 14, a second flow path 7b formed by branching from a middle portion of the first flow path 7a and connecting the sensing cuff 73 from the pump 14, and a third flow path 7c connecting the first flow path 7a to the atmosphere. Additionally, the first flow path 7a includes the first pressure sensor 17A. The first on-off valve 16A is provided between the first flow path 7a and the second flow path 7b. The second flow path 7b includes the second pressure sensor 17B. The second on-off valve 16B is provided between the first flow path 7a and the third flow path 7c.

In the fluid circuit 7 as described above, the first on-off valve 16A and the second on-off valve 16B are closed to connect only the first flow path 7a to the pump 14, thus fluidly connecting the pump 14, the palm-side cuff 71, and the back-side cuff 74. In the fluid circuit 7, the first on-off valve 16A is opened and the second on-off valve 16B is closed to connect the first flow path 7a and the second flow path 7b, thus fluidly connecting the pump 14, the palm-side cuff 71, and the back-side cuff 74, as well as the pump 14 and the sensing cuff 73. In the fluid circuit 7, the first on-off valve 16A is closed and the second on-off valve 16B is opened to connect the first flow path 7a and the third flow path 7c, thus fluidly connecting the palm-side cuff 71, the back-side cuff 74, and the atmosphere. In the fluid circuit 7, the first on-off valve 16A and the second on-off valve 16B are opened to connect the first flow path 7a, the second flow path 7b, and the third flow path 7c, thus fluidly connecting the palm-side cuff 71, the sensing cuff 73, the back-side cuff 74, and the atmosphere.

As illustrated in FIG. 21, the power supply unit 8 is provided in the recess 5d provided on the outer surface of the first end side of the curler 5 protruding from the device body 3. The power supply unit 8 is configured to be connectable to a connector provided at a tip of a charging cable of a charger, and fixable to the connector.

The power supply unit 8 includes a wiring portion 8a, a power supply terminal 8b, and a power supply cover 8c. The wiring portion 8a is accommodated in the recess 5d. The wiring portion 8a is connected to the power supply terminal 8b at one end, and to the control unit 55 at the other end. The power supply terminal 8b is accommodated in an end portion of the recess 5d on the side opposite to the outer case 31. The two power supply terminals 8b, each of which is constituted in, for example, a circular shape, are provided.

The power supply cover 8c is accommodated in the recess 5d. The power supply cover 8c is constituted in the same shape as that of the recess 5d and is fitted to the recess 5d. Further, the power supply cover 8c includes a hole that exposes each power supply terminal 8b to the outside. The power supply cover 8c and the wiring portion 8a are fixed in the recess 5d by, for example, an adhesive tape 8d.

The adhesive tape 8d is provided in, for example, regions between an inner surface of the power supply cover 8c and a surface of the wiring portion 8a, and between an inner surface of the recess 5d and the wiring portion 8a, to which a water-repellent treatment described later is not applied. Note that the locations where the adhesive tape 8d is provided are not limited.

The water-repellent portion 9 is provided on at least one of an inner surface of the flow path constituted by at least one of the case 11, the curler 5, or the cuff structure 6, or a portion of the outer surface of a member constituting the flow path, the portion being continuous with the flow path. The flow path is configured to communicate the inside and the outside of the case 11, and allow passage of air. The flow path is, for example, a ventilation hole constituted by at least one of the case 11, the curler 5, or the cuff structure 6, and a gap constituted by two of the case 11, the curler 5, and the cuff structure 6. In other words, the flow path conceptually includes an actively formed ventilation hole and gap between members.

The flow path is, for example, the hole 31c, a first gap S1 between the power supply cover 8c and the recess 5d, a second gap S2 between the cover portion 5a and the palm-side cuff 71, a third gap S3 between the cover portion 5a and the sensing cuff 73, and a fourth gap S4 between the cover portion 5a and the back-side cuff 74. The water-repellent portion 9 is provided in these flow paths.

Specifically, as illustrated in FIG. 21, the first gap S1 is a gap between the respective surfaces of the inner surface of the power supply cover 8c, the inner surface of the recess 5d, and the surfaces of the wiring portion 8a facing each other.

Specifically, as illustrated in FIG. 20, the second gap S2 includes a gap between an inner surface 5c11 of the first hole 5c1 of the cover portion 5a and an outer surface 84a of the connection portion 84, and a gap that is continuous with this gap and is between a surface 5a1 of the cover portion 5a on the living body side and a surface 83b of the tube 83 on the cover portion 5a side.

Specifically, the third gap S3 includes a gap between an inner surface 5c21 of the second hole 5c2 of the cover portion 5a and an outer surface 93a of the connection portion 93, and a gap that is continuous with this gap and is between the surface 5a1 of the cover portion 5a on the living body side and a surface 92b of the tube 92 on the cover portion 5a side.

Specifically, the fourth gap S4 includes a gap between an inner surface 5c31 of the third hole 5c3 of the cover portion 5a and an outer surface 103a of the connection portion 103, and a gap that is continuous with this gap and is between the surface 5a1 of the cover portion 5a on the living body side and the surface 101b of the bag structure 101 on the cover portion 5a side.

Specifically, the water-repellent portion 9 includes a first water-repellent portion 111 provided in the hole 31c as illustrated in FIG. 5, a second water-repellent portion 112 provided in the first gap S1 as illustrated in FIG. 21, and a third water-repellent portion 113 provided in the second gap S2, a fourth water-repellent portion 114 provided in the third gap S3, and a fifth water-repellent portion 115 provided in the fourth gap S4 as illustrated in FIG. 20.

Further, the water-repellent portion 9 is formed by applying a water-repellent treatment. The term “water-repellent treatment” refers to a treatment that increases a contact angle of water compared to that before the water-repellent treatment is applied. The water-repellent treatment is preferably a treatment that makes the contact angle of water 90 degrees or greater. As an example of the water-repellent treatment, a fluorine treatment is applied. A fluorine treatment is applied by, for example, applying a treatment liquid containing fluororesin using a brush or a cotton swab. The treatment liquid is dried, thereby forming a film containing the fluororesin. This film serves as the water-repellent portion. Alternatively, a member constituting the flow path may be formed from a material having a desired water repellency to constitute the water-repellent portion 9.

As illustrated in FIG. 4 and FIG. 5, the first water-repellent portion 111 includes a first inner water-repellent portion 111a provided on an inner surface of the hole 31c, and a first outer water-repellent portion 111b provided on a portion of the outer circumferential surface of the outer case 31 continuous with the hole 31c.

The first inner water-repellent portion 111a is provided, for example, in a region of the inner surface 31c1 that is continuous around an axis of the hole 31c. As a specific example, the first inner water-repellent portion 111a is provided across the entire region of the inner surface 31c1.

The first outer water-repellent portion 111b is provided around the second open end 31e of the outer circumferential surface 31f of the outer case 31. As a specific example, the first outer water-repellent portion 111b is provided by applying the water-repellent treatment to a region A having an annular shape around the second open end 31e and including an edge of the second open end 31e of the outer circumferential surface 31f.

As illustrated in FIG. 21, the second water-repellent portion 112 is provided by applying the water-repellent treatment to at least one of the inner surface of the power supply cover 8c, the inner surface of the recess 5d, or the surface of the wiring portion 8a constituting the first gap S1. The second water-repellent portion 112 is provided on, for example, a surface constituting an end portion of the first gap S1 on the case 11 side.

As a specific example, the second water-repellent portion 112 is provided by applying the water-repellent treatment to a region B, an end surface C, a region D, and portions of the surface of the wiring portion 8a facing the regions B and D. The region B is a region of an inner surface of the end portion of the recess 5d on the outer case 31 side and is continuous from a first end to a second end in the circumferential direction of the power supply cover 8c in a side view of the recess 5d from the longitudinal direction. The end surface C is a surface of the recess 5d on the outer case 31 side. The region D faces the region B on the inner surface of the end portion of the power supply cover 8c on the case 11 side.

The third water-repellent portion 113 includes a third inner water-repellent portion 113a and a third outer water-repellent portion 113b. As illustrated in FIG. 20, the third inner water-repellent portion 113a is provided by applying the water-repellent treatment to an inner surface of the first hole portion 5c1 and an outer surface of the connection portion 84.

As a specific example, the third inner water-repellent portion 113a is provided by applying the water-repellent treatment to a region E and a region F. The region E is a region of the inner surface of an end portion of the first hole portion 5c1 on the tube 83 side and is continuous around the axis of the first hole portion 5c1. The region F is a region of the outer surface of the connection portion 84 that faces the inner surface of the end portion of the first hole portion 5c1 and is continuous around the axis of the connection portion 84.

The third outer water-repellent portion 113b is provided around the first hole portion 5c1 on the surface 5a1 of the cover portion 5a on the living body side, and around the first hole portion 5c1 on the outer surface of the connection portion 84 and the outer surface of the tube 83. Specifically, the third outer water-repellent portion 113b is provided by applying a water-repellent treatment to a region G, a region H, and a region I. The region G is a region of the surface 5a1 of the cover portion 5a on the living body side, has an annular shape around the first hole portion 5c1, and includes an edge of the first hole portion 5c1. The region H is a region of the tube 83 having an annular shape and is continuous around the connection portion 84. The region I is a region of the end portion of the outer surface 84a of the connection portion 84 on the tube 83 side and is continuous around the axis of the connection portion 84. The water-repellent portion provided in the region H and the water-repellent portion provided in the region I are configured to be continuous.

Further, the third inner water-repellent portion 113a and the third outer water-repellent portion 113b are configured to be continuous. Specifically, the water-repellent portion of the third inner water-repellent portion 113a provided in the region E and the water-repellent portion of the third outer water-repellent portion 113b provided in the region G are configured to be continuous. The water-repellent portion of the third inner water-repellent portion 113a provided in the region F and the water-repellent portion of the third outer water-repellent portion 113b provided in the region H are configured to be continuous.

The fourth water-repellent portion 114 includes a fourth inner water-repellent portion 114a and a fourth outer water-repellent portion 114b. The fourth inner water-repellent portion 114a is provided around the second hole portion 5c2 on the surface 5a1 of the cover portion 5a on the living body side, and around the second hole portion 5c2 on an outer surface of the connection portion 93 and an outer surface of the tube 92.

As a specific example, the fourth inner water-repellent portion 114a is provided by applying the water-repellent treatment to a region J and a region K. The region J is a region of the inner surface of the end portion of the second hole portion 5c2 on the tube 92 side and is continuous around the axis of the second hole portion 5c2. The region K is a region of the outer surface 93a of the connection portion 93, faces the end portion of the second hole portion 5c2, and is continuous around the axis of the connection portion 93.

The fourth outer water-repellent portion 114b is provided around the second hole portion 5c2 on the surface 5a1 of the cover portion 5a on the living body side, and around the second hole portion 5c2 on the outer surface of the connection portion 93 and the outer surface of the tube 92. Specifically, the fourth outer water-repellent portion 114b is provided by applying the water-repellent treatment to a region L, a region M, and a region N. The region L is a region of the surface 5a1 of the cover portion 5a on the living body side, has an annular shape around the second hole portion 5c2, and includes an edge of the second hole portion 5c2. The region M is a region of the tube 92 having an annular shape and is continuous around the connection portion 93. The region N is a region of the end portion of the outer surface 93a of the connection portion 93 on the tube 92 side and is continuous around the axis of the connection portion 93. The water-repellent portion provided in the region M and the water-repellent portion provided in the region N are configured to be continuous.

Further, the fourth inner water-repellent portion 114a and the fourth outer water-repellent portion 114b are configured to be continuous. Specifically, the water-repellent portion of the fourth inner water-repellent portion 114a provided in the region K and the water-repellent portion of the fourth outer water-repellent portion 114b provided in the region N are configured to be continuous. The water-repellent portion of the fourth inner water-repellent portion 114a provided in the region J and the water-repellent portion of the fourth outer water-repellent portion 114b provided in the region L are configured to be continuous.

The fifth water-repellent portion 115 includes a fifth inner water-repellent portion 115a and a fifth outer water-repellent portion 115b. The fifth inner water-repellent portion 115a is provided by applying the water-repellent treatment to an inner surface of the third hole portion 5c3 and an outer surface of the connection portion 103.

As a specific example, the fifth inner water-repellent portion 115a is provided by applying the water-repellent treatment to a region O and a region P. The region O is a region of the inner surface of an end portion of the third hole portion 5c3 on the air bag 101 side and is continuous around the axis of the third hole portion 5c3. The region P is a region of the outer surface 103a of the connection portion 103, faces the inner surface of the end portion of the third hole portion 5c3, and is continuous around the axis of the connection portion 103.

The fifth outer water-repellent portion 115b is provided around the third hole portion 5c3 on the surface 5a1 of the cover portion 5a on the living body side, and around the third hole portion 5c3 on the outer surface of the connection portion 103 and the outer surface of the bag structure 101. Specifically, the fifth outer water-repellent portion 115b is provided by applying the water-repellent treatment to a region Q, a region R, and a region S. The region Q is a region of the surface 5a1 of the cover portion 5a on the living body side, has an annular shape around the third hole portion 5c3, and includes an edge of the third hole portion 5c3. The region R is a region of the air bag 101, has an annular shape, and is continuous around the connection portion 103. The region S is a region of the end portion of the outer surface 103a of the connection portion 103 on the air bag 101 side and is continuous around the axis of the connection portion 103. The water-repellent portion provided in the region R and the water-repellent portion provided in the region S are configured to be continuous.

Further, the fifth inner water-repellent portion 115a and the fifth outer water-repellent portion 115b are configured to be continuous. Specifically, the water-repellent portion of the fifth inner water-repellent portion 115a provided in the region P and the water-repellent portion of the fifth outer water-repellent portion 115b provided in the region S are configured to be continuous. The water-repellent portion of the fifth inner water-repellent portion 115a provided in the region O and the water-repellent portion of the fifth outer water-repellent portion 115b provided in the region Q are configured to be continuous.

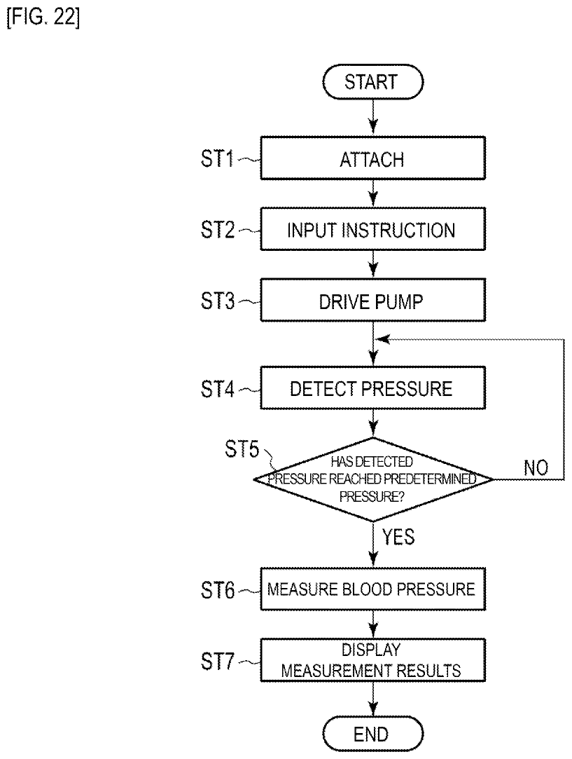

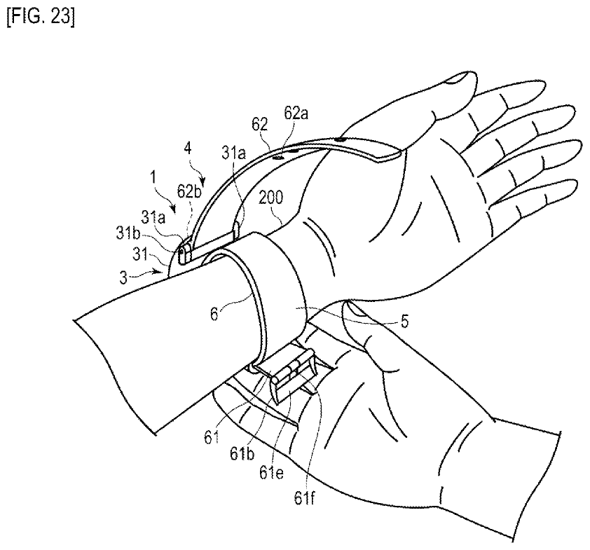

Next, an example of measurement of a blood pressure value using the blood pressure measurement device 1 will be described using FIG. 22 to FIG. 26. FIG. 22 is a flowchart illustrating an example of a blood pressure measurement using the blood pressure measurement device 1, illustrating both an operation of a user and an operation of the control unit 55. Additionally, FIG. 23 to FIG. 25 illustrate an example of the user attaching the blood pressure measurement device 1 to the wrist 200.

First, the user attaches the blood pressure measurement device 1 to the wrist 200 (step ST1). As a specific example, for example, the user inserts one of the wrists 200 into the curler 5, as illustrated in FIG. 23.

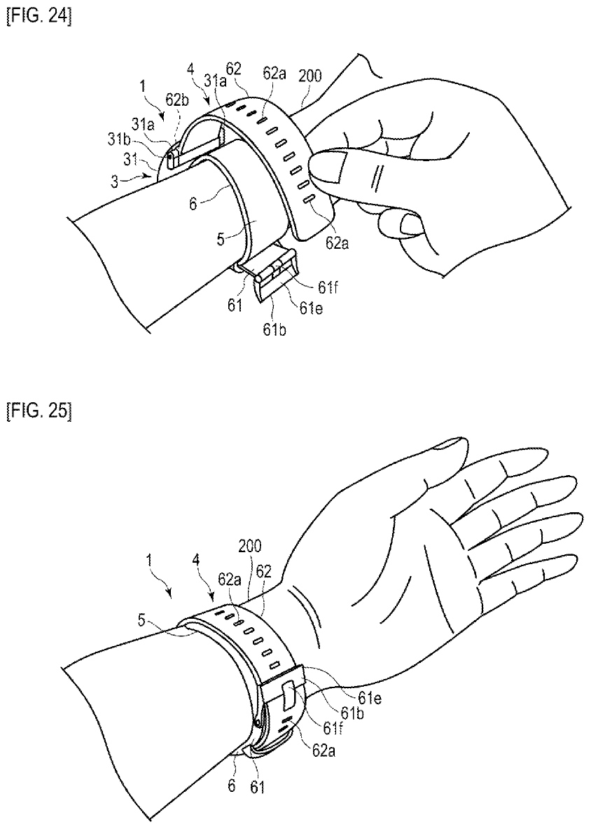

At this time, in the blood pressure measurement device 1, the device body 3 and the sensing cuff 73 are disposed at opposite positions of the curler 5, and thus the sensing cuff 73 is disposed in a region on the palm side of the wrist 200 in which the artery 210 resides. Thus, the device body 3 and the back-side cuff 74 are disposed on the hand back side of the wrist 200. Then, as illustrated in FIG. 24, the user passes the second belt 62 through the frame body 61e of the buckle 61b of the first belt 61 with the hand opposite to the hand on which the blood pressure measurement device 1 is disposed. The user then pulls the second belt 62 to bring the member on the inner circumferential surface side of the curler 5, that is, the cuff structure 6, into close contact with the wrist 200, and inserts the prong 61f into the small hole 62a. Thus, as illustrated in FIG. 25, the first belt 61 and the second belt 62 are connected, and the blood pressure measurement device 1 is attached to the wrist 200.

Then, the user operates the operation unit 13 to input an instruction corresponding to the start of measurement of the blood pressure value. The operation unit 13, on which an input operation of the instruction has been performed, outputs an electrical signal corresponding to the start of the measurement to the control unit 55 (step ST2). The control unit 55 receives the electrical signal, and then for example, opens the first on-off valve 16A, closes the second on-off valve 16B, and drives the pump 14 to feed compressed air to the palm-side cuff 71, the sensing cuff 73, and the back-side cuff 74 through the first flow path 7a and the second flow path 7b (step ST3). Thus, the palm-side cuff 71, the sensing cuff 73, and the back-side cuff 74 start to be inflated.

When the pump 14 is driven, the pump 14 draws air residing inside the case 11. Thus, the pressure inside the case 11 becomes negative. When the pressure inside the case 11 is negative, air flows from outside the case 11 to inside the case 11 through the hole 31c.

At this time, for water moving from outside the case 11 toward the hole 31c along with the air flowing into the hole 31c, adhesion of the water onto the periphery of the second open end 31e is suppressed by the first outer water-repellent portion 111b around the second open end 31e of the outer circumferential surface 31f of the outer case 31. Examples of the water here include sweat of the user and rain.

Furthermore, for water that is about to enter the hole 31c beyond the first outer water-repellent portion 111b, entry of the water is suppressed by a capillary phenomenon having an action of pushing the water from the hole 31c to outside the hole 31c, which is generated by the first inner water-repellent portion 111a.

Further, for water that is included in the air and reaches the moisture-permeable waterproof filter 36, entry of the water into the case 11 is suppressed by the moisture-permeable waterproof filter 36.

Furthermore, the hole 31c has a shape that is inclined with respect to the center line of the outer case 31. Thus, even in the unlikely event that the water enters the hole 31c when the user moves the wrist 200 to below the head of the user and sets the device in a posture in which the axial line of the outer case 31 is parallel to the gravitational direction in order to check the display unit 12, the water is moved to the second open end 31e side by gravitation.

For water that is about to enter the first gap S1, entry of the water into the first gap S1 is suppressed by a capillary phenomenon having an action of pushing the water from the first gap S1 to outside the first gap S1, which is generated by the second water-repellent portion 112.

For water that is about to enter the second gap S2, entry of the water into the second gap S2 is suppressed by a capillary phenomenon having an action of pushing the water from the second gap S2 to outside the second gap S2, which is generated by the third water-repellent portion 113.

For water that is about to enter the third gap S3, entry of the water into the third gap S3 is suppressed by a capillary phenomenon having an action of pushing the water from the third gap S3 to outside the third gap S3, which is generated by the fourth water-repellent portion 114.

For water that is about to enter the fourth gap S4, entry of the water into the fourth gap S4 is suppressed by a capillary phenomenon having an action of pushing the water from the fourth gap S4 to outside the fourth gap S4, which is generated by the fifth water-repellent portion 115.

The first pressure sensor 17A and the second pressure sensor 17B detect the pressures in the palm-side cuff 71, the sensing cuff 73, and the back-side cuff 74, and outputs, to the control unit 55, electrical signals corresponding to the pressures (step ST4). Based on the received electrical signals, the control unit 55 determines whether the pressures in the internal spaces of the palm-side cuff 71, the sensing cuff 73, and the back-side cuff 74 have reached a predetermined pressure for measurement of the blood pressure (step ST5). For example, in a case where the internal pressures of the palm-side cuff 71 and the back-side cuff 74 have not reached the predetermined pressure and the internal pressure of the sensing cuff 73 has reached the predetermined pressure, the control unit 55 closes the first on-off valve 16A and feeds compressed air through the first flow path 7a.

When the internal pressures of the palm-side cuff 71 and the back-side cuff 74 and the internal pressure of the sensing cuff 73 all have reached the predetermined pressure, the control unit 55 stops driving the pump 14 (YES in step ST5). At this time, as illustrated in FIG. 6, the palm-side cuff 71 and the back-side cuff 74 are sufficiently inflated, and the inflated palm-side cuff 71 presses the back plate 72. Additionally, the back-side cuff 74 presses the curler 5 in a direction away from the wrist 200, and then the belt 4, the curler 5, and the device body 3 move in a direction away from the wrist 200. As a result, the palm-side cuff 71, the back plate 72, and the sensing cuff 73 are pulled toward the wrist 200 side. In addition, when the belt 4, the curler 5, and the device body 3 move in a direction away from the wrist 200 due to the inflation of the back-side cuff 74, the belt 4 and the curler 5 move toward both lateral sides of the wrist 200, and the belt 4, the curler 5, and the device body 3 move in a state of close contact with both lateral sides of the wrist 200. Thus, the belt 4 and the curler 5, which are in close contact with the skin of the wrist 200, pull the skin on both lateral sides of the wrist 200 toward the hand back side. Note that the curler 5 may be configured to indirectly contact the skin of the wrist 200 via the sheet members 86 and 106, for example, as long as the curler 5 can pull the skin of the wrist 200.

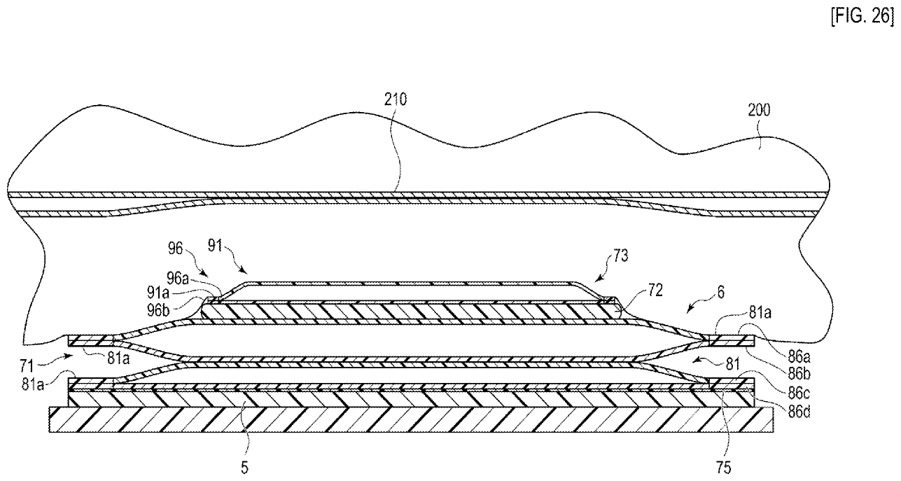

Further, the sensing cuff 73 is inflated by being fed with a predetermined amount of air such that the internal pressure reaches the pressure required to measure blood pressure, and is pressed toward the wrist 200 by the back plate 72 that is pressed by the palm-side cuff 71. Thus, the sensing cuff 73 presses the artery 210 in the wrist 200 and occludes the artery 210 as illustrated in FIG. 26.

Additionally, the control unit 55, for example, controls the second on-off valve 16B and repeats the opening and closing of the second on-off valve 16B, or adjusts the degree of opening of the second on-off valve 16B to pressurize the internal space of the palm-side cuff 71. In the process of pressurization, based on the electrical signal output by the second pressure sensor 17B, the control unit 55 obtains measurement results such as blood pressure values, for example, the systolic blood pressure and the diastolic blood pressure, and the heart rate and the like (step ST6). The control unit 55 outputs an image signal corresponding to the obtained measurement results to the display unit 12, and displays the measurement results on the display unit 12 (step ST7). In addition, after the end of the blood pressure measurement, the control unit 55 opens the first on-off valve 16A and the second on-off valve 16B.

The display unit 12 receives the image signal, and then displays the measurement results on the screen. The user views the display unit 12 to confirm the measurement results. Note that, after the end of the measurement, the user removes the prong 61f from the small hole 62a, removes the second belt 62 from the frame body 61e, and removes the wrist 200 from the curler 5, thus removing the blood pressure measurement device 1 from the wrist 200.

With the blood pressure measurement device 1 according to the present embodiment configured in this way, the water-repellent portion 9 is provided, making it possible to suppress the entry of water into the case 11 through a flow path such as the hole 31c, the first gap S1, the second gap S2, the third gap S3, and the fourth gap S4 provided in the case 11. This improves the water resistance of the blood pressure measurement device 1.

Specifically, the blood pressure measurement device 1 can suppress the adherence of water around the second open end 31e of the outer circumferential surface 31f of the outer case 31 by the first outer water-repellent portion 111b of the first water-repellent portion 111, and thus suppress the entry of water into the hole 31c. As a result, because the entry of water into the case 11 through the hole 31c is suppressed, the water resistance of the blood pressure measurement device 1 can be improved.

Furthermore, the blood pressure measurement device 1 utilizes a capillary phenomenon having the action of pushing water that is about to enter the hole 31c to outside the hole 31c, which is generated by the first inner water-repellent portion 111a of the first water-repellent portion 111, thereby suppressing the entry of water into the hole 31c. Thus, the water resistance of the blood pressure measurement device 1 can be improved.

Furthermore, the second water-repellent portion 112 is provided in the first gap 51, the third water-repellent portion 113 is provided in the second gap S2, the fourth water-repellent portion 114 is provided in the third gap S3, and the fifth water-repellent portion 115 is provided in the fourth gap S4. Thus, a capillary phenomenon having an action of pushing water that is about to enter each of the gaps to outside the gap, which is generated by the corresponding water-repellent portion, can be utilized to suppress the entry of water into the gap. Thus, the water resistance of the blood pressure measurement device 1 can be improved.

Furthermore, in the blood pressure measurement device 1, the second open end 31e of the hole 31c disposed on the outer circumferential surface 31f of the outer case 31 is disposed further on the living body side in the axial direction of the outer case 31 than the first open end 31d disposed on the inner circumferential surface of the outer case 31. Thus, in a state in which the axial direction of the outer case 31 is parallel to the gravitational direction when, for example, the user checks the display unit, the second open end 31e is disposed below the first open end 31d. Accordingly, even in the unlikely event that water enters the hole 31c, the water that enters the hole 31c can be removed from the second open end 31e to outside the case 11 by gravitation.

Furthermore, the hole 31c is configured as a hole having a linear shape inclined with respect to the axial direction of the outer case 31. Thus, even in the unlikely event that water enters the hole 31c, the blood pressure measurement device 1 easily moves this water toward the second open end 31e.

Furthermore, the blood pressure measurement device 1 includes the moisture-permeable waterproof filter 36 at the first open end 31d of the hole 31c. Thus, even in a case where water enters the hole 31c, the entry of the water into the case 11 can be suppressed by the moisture-permeable waterproof filter 36. Thus, the water resistance of the blood pressure measurement device 1 can be improved.

Furthermore, the first water-repellent portion 111 is provided in the hole 31c provided for air intake. Thus, the blood pressure measurement device 1 can suppress the entry of water into the case 11 through the hole 31c even if the performance of regulating the passage of water required for the moisture-permeable waterproof filter 36 is reduced compared to a case where the first water-repellent portion 111 is not provided. As a result, it is possible to use the moisture-permeable waterproof filter 36 that does not strictly regulate the passage of air while satisfying the water passage regulation performance required for the blood pressure measurement device 1. Thus, when the pressure inside the case 11 becomes negative due to the driving of the pump 14, air can smoothly flow through the holes 31c, making it possible to suppress excessive negative pressure inside the case 11.

Furthermore, the water-repellent portion 9 is, as an example, provided by applying a fluorine treatment. Thus, the blood pressure measurement device 1 does not increase in size due to the water-repellent portion 9. Furthermore, because the water-repellent portion 9 is provided by applying a fluorine treatment, an increase in the number of components of the blood pressure measurement device 1 can be prevented. Furthermore, because no component is required to configure the water-repellent portion 9, the design of the blood pressure measurement device 1 does not need to be changed.

Note that the present invention is not limited to the embodiments described above. As an example, a configuration has been described in which the first water-repellent portion 111 is provided on the inner surface of the hole 31c serving as a flow path and on a portion around the second open end 31e on the outer circumferential surface 31f of the outer case 31, the portion being continuous with the hole 31c. However, the configuration is not limited thereto. In another example, the first water-repellent portion 111 may be configured to be provided only on the inner surface of the hole 31c, or may be configured to be provided only in a portion of the outer circumferential surface 31f that is continuous with the second open end 31e.

Further, while a configuration in which the second water-repellent portion 112 is provided only on the inner surface of the first gap S1 serving as a flow path has been described as an example, the configuration is not limited thereto. In another example, the second water-repellent portion 112 may be configured to be further provided on a portion of the outer surface of the curler 5 that is continuous with the first gap S1.

Further, in the present embodiment, as one example of a flow path configured by at least one of the case 11, the cuff structure 6, or the curler 5 that is provided with the water-repellent portion 9, communicates the inside and the outside of the case 11, and allows flow of air, the hole 31c formed in the outer case 31, the first gap S1 constituted by the curler 5, the power supply cover 8c of the power supply unit 8, and the wiring portion 8a, and the second gap S2, the third gap S3, and the fourth gap S4 constituted by the cover portion 5a and the cuff structure 6 has been described. Nevertheless, the flow path is not limited to these. In another example, the water-repellent portion may be provided on at least one of the inner surface of the gap between the windshield 32 and the outer case 31 or a portion of the outer circumferential surface 31f of the outer case 31 that is continuous with the gap. The water-repellent portion 9 can be provided in a flow path in which water may enter the case 11.

Further, in the example described above, the blood pressure measurement device 1 improves the waterproof performance by the water-repellent portion 9.

Nevertheless, the blood pressure measurement device 1 may further include a hydrophilic portion around the water-repellent portion provided on a portion of the outer surface of a member constituting the flow path, the portion being continuous with the flow path. The hydrophilic portion is a portion having higher affinity to water compared to an area where the hydrophilic portion is not provided. Alternatively, the hydrophilic portion is a portion having a desired affinity.

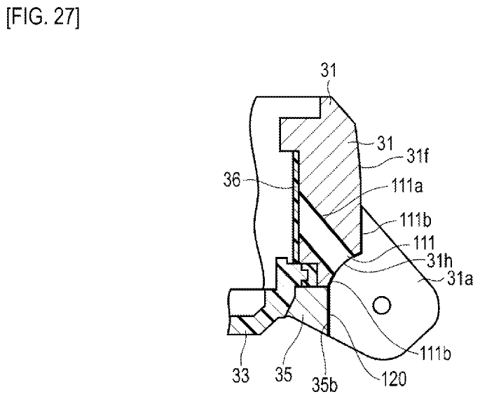

Now, a modified example of the blood pressure measurement device 1 including the hydrophilic portion will be described using FIG. 27. FIG. 27 is a cross-sectional view of the hole 31c of the outer case 31 of the blood pressure measurement device 1. As illustrated in FIG. 27, the blood pressure measurement device 1 further includes a hydrophilic portion 120. The hydrophilic portion 120 is provided by applying hydrophilic treatment to a portion of the outer circumferential surface 35b of the back cover 35 that is adjacent to the first outer water-repellent portion 111b. In this modified example, the hydrophilic portion 120 is disposed below the first outer water-repellent portion 111b, as an example. Here, “below the first outer water-repellent portion 111b” refers to a portion below the first outer water-repellent portion 111b in a posture of the blood pressure measurement device 1 in which the center line of the outer case 31 is parallel to the gravitational direction and the cover portion 5a is disposed below the windshield 32. Alternatively, the hydrophilic portion 120 may be constituted in an annular shape surrounding the first outer water-repellent portion 111b.

The hydrophilic treatment is, for example, a surface treatment such as a plasma treatment. Alternatively, the hydrophilic treatment is a treatment of applying a hydrophilic material using a brush, swab, or the like. Alternatively, the hydrophilic portion 120 may be formed by forming a portion to be provided with the hydrophilic portion 120 from a material having the desired hydrophilicity. Alternatively, the hydrophilic portion 120 may be formed by forming only a portion to be provided with the hydrophilic portion 120 from a material having the desired hydrophilicity. Alternatively, the hydrophilic portion 120 may be formed by configuring the entire member to be provided with the hydrophilic portion using a member having the desired water repellency and applying the hydrophilic treatment to the member having the water repellency, or by providing a member having the desired hydrophilicity. The hydrophilic portion 120 is configured to be continuous with the first outer water-repellent portion 111b, for example.

In this modified example, the hydrophilic portion 120 enables water moved by the first outer water-repellent portion 111b and water moving from outside the first outer water-repellent portion 111b toward the first outer water-repellent portion 111b to be retained in the hydrophilic portion 120. By making it possible to retain water in the hydrophilic portion 120, it is possible to suppress the entry of water into the hole 31c and thus improve the waterproof performance of the blood pressure measurement device 1.

Note that, in the example described above using FIG. 27, a configuration in which the hydrophilic portion 120 is provided around the first outer water-repellent portion 111b has been described as an example. Nevertheless, the hydrophilic portion 120 is not limited to being provided around the first outer water-repellent portion 111b.

In the case of a configuration in which the flow path, for instance, the hole 31c and the gaps S1, S2, S3, and S4, is constituted by at least one of the case 11, the cuff structure 6, or the curler 5, and the water-repellent portion 9 is provided on at least one of an inner surface of the flow path or a portion of an outer surface of the at least one of the case 11, the cuff structure 6, or the curler 5 that constitutes the flow path, the portion being continuous with the flow path, the hydrophilic portion may be provided around the water-repellent portion 9 provided on the outer surface of the at least one of the case 11, the cuff structure 6, or the curler 5 that constitutes the flow path. As another example, the hydrophilic portion may be provided on the surface 5a1 of the cover portion 5a of the curler 5 on the living body side, around the water-repellent portion provided in the region G of the third outer water-repellent portion 113b of the third water-repellent portion 113b illustrated in FIG. 21. Further, in another example, the hydrophilic portion may be provided around the water-repellent portion provided in the region H of the tube 83, which is an example of the cuff structure 6. Further, in another example, the hydrophilic portion, as illustrated in FIG. 21, may be provided around the second water-repellent portion 112 on the surface of the curler 5.

Further, in the examples described above, the blood pressure measurement device 1 is provided with the water-repellent portion on at least one of the inner surface of the flow path communicating the inside and the outside of the case 11 or a portion of the outer surface of a member constituting the flow path, the portion being continuous with the flow path. Thus, the water resistance is improved.

Nevertheless, the water-repellent portion is not limited to being provided only on at least one of the inner surface of the flow path or a portion of the outer surface of a member constituting the flow path, the portion being continuous with the flow path. For example, the water-repellent portion 9 may be provided in a gap other than a gap constituting the flow path communicating the inside and the outside of the case 11. As an example, the water-repellent portion 9 may be provided in a gap between the belt 4 and the curler 5. With the water-repellent portion provided on a surface constituting such a gap, it is possible to suppress the pooling of water in the gap, which is preferable from the perspective of sanitation.

Further, in the examples described above, the curler 5 includes the cover portion 5a, and the back lid that covers the outer case 31 on the living body side is constituted by the cover portion 5a and the back cover 35, but no such limitation is intended. That is, a configuration may be adopted in which the blood pressure measurement device 1 includes a back lid that covers the outer case 31 on the living body side without including the back cover 35 and the cover portion 5a, and the curler 5 is fixed to the back lid.

Further, in the examples described above, the configuration including the two on-off valves 16 of the first on-off valve 16a and the second on-off valve 16b has been described, but no such limitation is intended. For example, a configuration may be adopted in which four on-off valves 16 are provided.

For example, the timings when the first on-off valve 16A and the second on-off valve 16B are opened and closed during blood pressure measurement by the blood pressure measurement device 1 are not limited to the timings in the examples described above, and can be set as appropriate. Additionally, the example has been described in which the blood pressure measurement device 1 performs blood pressure measurement by calculating the blood pressure from the pressure measured during the process of pressurizing the palm-side cuff 71. However, no such limitation is intended and the blood pressure may be calculated during the depressurization process or during both the pressurization process and the depressurization process.

Additionally, in the examples described above, the configuration has been described in which the back plate 72 includes the plurality of grooves 72a, but no such limitation is intended. For example, for control of deformability and the like, the number, the depth, and the like of the plurality of grooves 72a may be set as appropriate, and the back plate 72 may be configured to include a member that suppresses deformation.

Further, in the examples described above, the configuration in which the blood pressure measurement device 1 provided with the water-repellent section 9 includes the curler 5 has been described. That is, the configuration has been described in which the flow path, for instance, the hole 31c and the gaps S1, S2, S3, and S4, is constituted by at least one of the case 11, the cuff structure 6, or the curler 5, and the water-repellent portion 9 is provided on at least one of an inner surface of the flow path or a portion of an outer surface of the at least one of the case 11, the cuff structure 6, or the curler 5 that constitutes the flow path, the portion being continuous with the flow path. Nevertheless, as another example, a configuration may be adopted in which the blood pressure measurement device 1 does not include the curler 5.

When the blood pressure measurement device 1 has a configuration in which the curler 5 is not included, the flow path need only be constituted by at least one of the case 11 or the cuff structure 6 and configured to communicate the inside and the outside of the case 11 and allow flow of air. Examples of the flow path include a ventilation hole constituted by at least one of the case 11 or the cuff structure 6, and a gap constituted by the two members of the case 11 and the cuff structure 6. In this way, when the blood pressure measurement device 1 has a configuration in which the curler 5 is not included, the water-repellent portion 9 need only be provided on at least one of the inner surface of the flow path or a portion of the outer surface of the at least one of the case 11 or the cuff structure 6 that constitutes the flow path, the portion being continuous with the flow path.

Further, in the examples described above, the blood pressure measurement device 1 provided with the water-repellent portion 9 has been described using an example of an electronic blood pressure measurement device having an aspect of a wearable device attached to the wrist 200 of the living body, but no such limitation is intended. For example, the blood pressure measurement device may be a so-called upper arm blood pressure measurement device having an aspect of being wrapped around the upper arm at the time of blood pressure measurement rather than an aspect of being continually attached to the user.

The upper arm blood pressure measurement device attached at the time of use has a configuration in which the curler is not included. Even with the upper arm blood pressure measurement device having a configuration in which the curler is not included, the flow path need only be constituted by at least one of the case 11 or the cuff structure 6 and configured to communicate the inside and the outside of the case 11 and allow flow of air. Examples of the flow path include a ventilation hole constituted by at least one of the case 11 or the cuff structure 6, and a gap constituted by the two members of the case 11 and the cuff structure 6.

Further, in the examples described above, when the flow path, for instance, the hole 31c and the gaps S1, S2, S3, and S4, is constituted by at least one of the case 11, the cuff structure 6, or the curler 5, the hydrophilic portion 120 is provided around the water-repellent portion provided on the outer surface of the at least one of the case 11, the cuff structure 6, or the curler 5.

Nevertheless, when the blood pressure measurement device 1 is configured to not include the curler 5 as described above and the flow path is constituted by at least one of the case 11 or the cuff structure 6, the hydrophilic portion need only be provided around the water-repellent portion provided on the outer surface of the at least one of the case 11 or the cuff structure 6 that constitutes the flow path.

In other words, the embodiments described above are mere examples of the present invention in all respects. Of course, various modifications and variations can be made without departing from the scope of the present invention. Thus, specific configurations in accordance with an embodiment may be adopted as appropriate at the time of carrying out the present invention.

### REFERENCE SIGNS LIST

1 Blood pressure measurement device
3 Device body
4 Belt
5 Curler
5a Cover portion
5b Screw hole
5c Hole portion
6 Cuff structure
7 Fluid circuit
7a First flow path
7b Second flow path
7c Third flow path
11 Case
12 Display unit
13 Operation unit
14 Pump
15 Flow path unit
16 On-off valve
16A First on-off valve
16B Second on-off valve
17 Pressure sensor
17A First pressure sensor
17B Second pressure sensor
18 Power supply unit
19 Vibration motor
20 Control substrate
31 Outer case
31a Lug
31b Spring rod
32 Windshield
33 Base
35 Back cover
35a Screw
41 Button
42 Sensor
43 Touch panel
51 Substrate
52 Acceleration sensor
53 Communication unit
54 Storage unit
55 Control unit
56 Main CPU
57 Sub-CPU
61 First belt
61a Belt portion
61b Buckle
61c First hole portion
61d Second hole portion
61e Frame body
61f Prong
62 Second belt
62a Small hole
62b Third hole portion
71 Palm-side cuff (cuff)
72 Back plate
72a Groove
73 Sensing cuff
74 Back-side cuff (cuff)
81 Air bag (bag-like structure)
84 Connection portion
86, 86A Sheet member
86a First sheet member
86b Second sheet member
86b1 Opening
86c Third sheet member
86c1 Opening
86d Fourth sheet member
91 Air bag (bag-like structure)
92 Tube
93 Connection portion
96 Sheet member
96a Fifth sheet member
96b Sixth sheet member
101 Air bag (bag-like structure)
103 Connection portion
106, 106A Sheet member
106a Seventh sheet member
106b Eighth sheet member
106b1 Opening
106c Ninth sheet member
106c1 Opening
106d Tenth sheet member
106d1 Opening
106e Eleventh sheet member
106e1 Opening
106f Twelfth sheet member
106f1 Opening
106 g Thirteenth sheet member
106g1 Opening
106 h Fourteenth sheet member
106h1 Opening
106i Fifteenth sheet member
106i1 Opening
106j Sixteenth sheet member
106j1 Opening
106k Seventeenth sheet member
106k1 Opening
106l Eighteenth sheet member
111 First water-repellent portion
111a First inner water-repellent portion
111b First outer water-repellent portion
112 Second water-repellent portion
113 Third water-repellent portion
113a Third inner water-repellent portion
113b Third outer water-repellent portion
114 Fourth water-repellent portion
114a Fourth inner water-repellent portion
114b Fourth outer water-repellent portion
115 Fifth water-repellent portion
115a Fifth inner water-repellent portion
115b Fifth outer water-repellent portion
200 Wrist
210 Artery

1. A blood pressure measurement device comprising:
a case;
a cuff structure connected to the case and configured to be inflated with a fluid;
a flow path constituted by at least one of the case or the cuff structure, the flow path being configured to communicate an inside and an outside of the case and allow flow of air; and
a water-repellent portion provided on at least one of an inner surface of the flow path or a portion of an outer surface of the at least one of the case or the cuff structure that constitutes the flow path, the portion being continuous with the flow path, wherein
the case includes an outer case having a tubular shape, and
the flow path is a ventilation hole including a first open end and a second open end, the first open end being disposed on an inner circumferential surface of the outer case, the second open end being disposed on an outer circumference of the outer case, the second open end being disposed on a living body side in an axial direction of the outer case with respect to the first open end.

2. The blood pressure measurement device according to claim 1, further comprising:
a pump accommodated inside the case and configured to compress air inside the case and feed the air to the cuff structure.

3. The blood pressure measurement device according to claim 1, further comprising:
a moisture-permeable waterproof filter provided at the first open end and configured to allow passage of air and to regulate passage of water.

4. The blood pressure measurement device according to claim 1, further comprising:
a hydrophilic portion provided around the water-repellent portion provided on the outer surface of the at least one of the case or the cuff structure that constitutes the flow path.

5. The blood pressure measurement device according to claim 1, further comprising:
a curler curving to follow a circumferential direction of a site of a living body where the curler is attached, the curler being formed with a first end and a second end spaced apart from each other, the cuff structure being provided on the curler, wherein
the flow path is constituted by at least one of the case, the cuff structure, or the curler, and
the water-repellent portion is provided on at least one of an inner surface of the flow path or a portion of an outer surface of the at least one of the case, the cuff structure, or the curler that constitutes the flow path, the portion being continuous with the flow path.

6. The blood pressure measurement device according to claim 5, further comprising:
a hydrophilic portion provided around the water-repellent portion provided on the outer surface of the at least one of the case, the cuff structure, or the curler that constitutes the flow path.
